(19) United States
(12) Patent Application Publication
(10) Pub. No.: US 2023/0180725 A1
(43) Pub. Date: Jun. 15, 2023

(54) Production of Human Cells, Tissues, and Organs in a Growth Factor Receptor-Deficient Animal Host

(71) Applicants: The Board of Trustees of the Leland Stanford Junior University, Stanford, CA (US); The University of Tokyo, Bunkyo City (JP)

(72) Inventors: Toshiya Nishimura, Redwood City, CA (US); Hiromitsu Nakauchi, Woodside, CA (US); Motoo Watanabe, Bunkyo City (JP)

(21) Appl. No.: 17/995,742

(22) PCT Filed: Apr. 8, 2021
(86) PCT No.: PCT/US2021/026380

§ 371 Date: Oct. 7, 2022

Related U.S. Application Data
(60) Provisional application No. 63/008,358, filed on Apr. 10, 2020.

(51) Int. Cl. A01K 67/027; C12N 15/90; C12N 15/11; C12N 9/22

(52) U.S. Cl. CPC A01K 67/0275; C12N 15/907; C12N 15/11; C12N 9/22; C12N 2310/20; C12N 2800/80; A01K 2227/105; A01K 2217/075; A01K 2207/12; A01K 2267/025

(57) ABSTRACT

Methods of generating functional human organs and tissue in animal bodies suitable for transplantation into human subjects are provided. In particular, the contribution of human donor cells to tissues and organs can be increased in interspecies host embryos by knocking out a growth factor receptor gene such as the insulin-like growth factor 1 receptor or insulin receptor gene. Almost entirely donor-derived functional organs and tissue can be generated by using this method. The methods described herein are useful for generating human organs and tissue in animals and may be helpful for overcoming the current problems with organ shortage for transplantation therapy. Additionally, such organs and tissue can be used in drug discovery, drug screening, and toxicology testing.

### BACKGROUND OF THE INVENTION

The generation of human organs in an animal body by employing a vacant organ niche is a brand-new concept to solve the organ shortage in transplantation therapy. Although there are several reports of the generation of human-animal chimeras, a human organ has not yet been generated within an animal body. One of the major problems is the low contribution of human cells to animal tissues. Based on reports, the contribution of human cells to animal tissue is about 1 human cell in 100,000 animal cells. Since sufficient cell contribution is necessary for fully functional human organ regeneration, enhancement of donor cell contribution would provide significant benefit to further efforts to regenerate functional human organs for transplantation.

### SUMMARY OF THE INVENTION

Blastocyst injection is a conventional method to generate chimeric animals by injecting stem cells into embryos. Generally, injection of stem cells into a blastocyst results in a chimeric animal in which both donor and host cells exist in various tissues or organs (i.e., cellular mosaic). Methods are provided for increasing the donor cell contribution to host organs by knocking out a growth factor receptor gene such as the insulin-like growth factor 1 receptor (IGF1 R) or insulin receptor (INR, also known as INSR) gene in interspecies host embryos. The inventors have shown that the donor contribution to multiple organs can be increased by knockout of the IGF1 R or INR gene. In some cases, the human donor cell contribution to host kidneys, lungs, thymus, heart, and brain in adult-stage chimeric animals has exceeded 95% (see Examples). Generation of functional organs in animal bodies suitable for transplantation into human subjects will provide an alternative for dealing with the problem of chronic organ shortage for transplantation therapy. Additionally, such organs can be used in drug discovery, drug screening, and toxicology testing.

In one aspect, a method of creating a chimeric organ or tissue donor is provided, the method comprising: a) genetically modifying a non-human animal host embryo by deleting or inactivating a growth factor receptor gene; and b) transplanting mammalian stem cells having a wild-type growth factor receptor gene into the non-human animal host embryo, wherein chimeric organs and tissue comprising mammalian cells are produced from the mammalian stem cells as the non-human animal host embryo grows.

In certain embodiments, the growth factor receptor gene is IGF1 R or INR.

In certain embodiments, the non-human animal is a vertebrate including, without limitation, a mammal.

In certain embodiments, the non-human host animal embryo is at the blastocyst stage or morula stage.

In certain embodiments, the stem cells are embryonic stem cells, adult stem cells, or induced pluripotent stem cells. In some embodiments, the stem cells are human stem cells.

In certain embodiments, the mammalian stem cells are genetically modified to overexpress the growth factor receptor gene.

In certain embodiments, the transplantation of the mammalian stem cells is performed in utero to a conceptus or to the embryo in in vitro culture. In certain embodiments, the transplantation of the mammalian cells is performed when the non-human host animal embryo is at the blastocyst stage or morula stage.

In another aspect, a chimeric organ or tissue donor, produced by the methods described herein, is provided.

In another aspect, a method of transplanting an organ or tissue into a mammalian recipient subject is provided, the method comprising: a) creating a chimeric organ or tissue donor according to a method described herein; and b) transplanting a chimeric organ, tissue, or cells from the donor to the mammalian recipient subject.

In certain embodiments, the mammalian stem cells transplanted into the non-human animal host embryo are induced pluripotent stem cells derived from cells from the mammalian recipient subject. In other embodiments, the mammalian stem cells are adult stem cells from the mammalian recipient subject.

In certain embodiments, the mammalian recipient subject is human.

In certain embodiments, at least 90% of the cells in the chimeric organ or tissue are produced from the mammalian stem cells.

In certain embodiments, the stem cells are human stem cells.

In certain embodiments, the chimeric organ or tissue is a kidney, a lung, a heart, an intestine, a pancreas, a thymus, a liver, kidney tissue, lung tissue, heart tissue, intestine tissue, pancreas tissue, thymus tissue, liver tissue, or connective tissue.

In certain embodiments, the method further comprises administering an immunosuppressive agent to the mammalian recipient subject.

In another aspect, a non-human animal host embryo is provided comprising: a) a genetically modified genome comprising a knockout of an insulin-like growth factor 1 receptor (IGF1R) gene or an insulin receptor (INR) gene; and b) transplanted mammalian stem cells having a wild-type growth factor receptor gene, wherein said non-human animal host embryo produces chimeric organs and tissue comprising mammalian cells from the mammalian stem cells during development.

In certain embodiments, the non-human animal host embryo is a vertebrate. In some embodiments, the vertebrate is a mammal.

In certain embodiments, the non-human animal host embryo is at the blastocyst stage or morula stage.

In certain embodiments, the mammalian stem cells transplanted into the non-human animal host embryo are embryonic stem cells, adult stem cells, or induced pluripotent stem cells.

In certain embodiments, the mammalian stem cells transplanted into the non-human animal host embryo are human stem cells.

In certain embodiments, the mammalian stem cells are genetically modified to overexpress the IGF1R gene or the INR gene.

In certain embodiments, the knockout comprises a deletion of the IGF1R gene or the INR gene or a frameshift mutation in the IGF1R gene or the INR gene. In some embodiments, both alleles of the IGF1R gene or the INR gene are knocked out in the non-human animal host embryo.

In another aspect, a use of the non-human animal host embryo, described herein, in the manufacture of a chimeric mammalian organ or tissue is provided.

In certain embodiments, at least 90% of the cells in the chimeric organ or tissue are derived from the mammalian stem cells transplanted into the non-human animal host embryo.

In certain embodiments, the mammalian stem cells transplanted into the non-human animal host embryo are human stem cells.

In another aspect, a method of transplanting an organ or tissue into a mammalian subject is provided, the method comprising transplanting a chimeric organ or tissue produced from a non-human animal host embryo, described herein, to the mammalian subject.

In certain embodiments, at least 90% of the cells in the chimeric organ or tissue are produced from the mammalian stem cells transplanted into the non-human animal host embryo.

In certain embodiments, the mammalian stem cells transplanted into the non-human animal host embryo are human stem cells.

### BRIEF DESCRIPTION OF THE DRAWINGS

FIGS. 1A-1F: Donor Cells Predominantly Proliferate in Igf1r Null Embryos Generated Using the CRISPR/Cas9 and sgRNA Complex. (FIG. 1A) Targeting locus of sgRNA1 in mouse Igf1r. (FIG. 1B) Representative images of Igf1r null (Null) and wild-type (WT) neonates at E18.5. (FIG. 1C) Body weight and length of Igf1r null (n=10) and WT (n=4) at E18.5. Means±SDs. Statistical significance was calculated by Mann-Whitney U testing. **p<0.005 and ***p<0.0005. (FIG. 1D) Experimental flow for generating Igf1r null and WT chimeras. (FIG. 1E) Chimerism, Igf1r, or WT chimeras (n=6 chimeras per group) in blood and connective tissue (CT) at E11.5 measured by flow cytometry. Means±SDs. (FIG. 1F) CT:blood chimerism ratio of Igf1r null or WT chimeras at E10.5 (n=3 Igf1r null, n=4 WT chimeras per group), E11.5 (n=3 Igf1r null, n=4 WT chimeras per group), E12.5 (n=3 Igf1r null, n=7 WT chimeras per group), and E18.5 (n=7 Igf1r null, n=3 WT chimeras per group). Means±SDs. Statistical significance was calculated by Mann-Whitney U testing (WT versus Igf1r null). *p<0.05.

FIGS. 2A-2F. Increased Organ and Tissue Chimerism in Igf1r Null Chimeras from Fetal to Neonatal Stages (FIG. 2A) Experimental flow of organ chimerism analysis using the ddPCR platform. (FIG. 2B-2E) Comparisons of organ:blood chimerism (kidney, lung, brain) and CT:blood chimerism in Igf1r null chimeras (n=3 at E11.5, n=7 at E18.5 chimeras per group) with WT chimeras (n=4 at E11.5, n=3 at E18.5 chimeras per group). Statistical significance was calculated by Mann-Whitney U testing (Igf1r null versus WT). *p<0.05. (FIG. 2F) GFP expression and macroscopic appearance of an Igf1r null (right) and WT (non-chimera) (left) at E18.5. BF: bright field.

FIGS. 3A-3I. Dissection and Characterization of Adult Igf1r Null Chimeras. (FIG. 3A) Igf1r null chimera aged 3 weeks. *Igf1r null chimera, C: WT (non-chimera), W: WT chimera. (FIG. 3B) Kidney:blood chimerism ratio of Igf1r null (n=4) and WT chimeras (n=7). Means±SDs. Statistical significance was calculated by Mann-Whitney U testing. *p<0.05. (FIG. 3C) Median chimerism, kidneys of Igf1r null chimeras. Statistical significance was calculated by Mann-Whitney U testing. *p<0.05. (FIG. 3D) Macroscopic appearance and GFP expression in kidneys of Igf1r null and WT chimeras. (FIG. 3E) Microscopic appearance in kidneys of Igf1r null chimera and WT mouse (control). Scale bar: 200 μm. (FIGS. 3F and 3G) Median blood urea nitrogen (BUN) and creatinine (CRE) concentrations in the sera of Igf1r null chimeras (BUN: n=4, CRE: n=3) and WT mice (n=3, control). (FIGS. 3H and 31) GFP expression (green) and immunohistochemical staining (red) of adult kidneys of Igf1r null chimera (Igf1r null, n=4), WT chimera (Igf1r WT, n=4), and WT mouse (control, n=4) for GFP (green) with antibodies against specific renal components nephrin for podocytes and calbindin for collecting ducts in renal medulla. Nuclei were stained with DAPI (gray). Scale bars: 50 μm (left panel), 25 μm (right). Arrowheads, co-staining of GFP with specific markers in Igf1r chimera. Dotted lines, enlarged areas presented in panels at right.

FIGS. 4A-4J. Dissection and Characterization of Interspecies Igf1r Null Chimera Neonate. (FIG. 4A) Experimental flow for generating interspecies Igf1r null chimeras. (FIG. 4B) EGFP expression (rat cells), interspecies rat-mouse Igf1r null chimera at E18.5. Scale bar: 1 mm. (FIG. 4C) Macroscopic appearance, interspecies rat-mouse Igf1r null chimera (upper), and Igf1r null mouse (lower) at E18.5. (FIG. 4D) Whole-body chimerism, interspecies rat-mouse Igf1r null chimeras (Igf1r null, n=6), and interspecies rat-mouse WT chimeras at E18.5 (WT, n=7 chimeras). Means±SDs. Statistical significance was calculated by unpaired 2-tailed t testing. *p<0.05. (FIG. 4E-41) Chimerism of each organ or tissue (kidney, lung, heart, thymus, and CT) in interspecies rat-mouse Igf1r null chimeras (Igf1r null, n=6 chimeras per group) or interspecies rat-mouse WT chimeras at E18.5 (WT, n=7 chimeras per group). Means±SDs. Statistical significance was calculated by unpaired 2-tailed t testing. *p<0.05 and ***p<0.0005. (FIG. 4J) Schematic representation of the cell-competitive niche.

FIGS. 5A-5B: The organ chimerism increase in Inr chimera at E18.5. FIG. 5A. The whole per blood chimerism ratio of Inr knockout (Inr KO, n=4 chimeras per group) chimera or wild-type chimera (WT, n=5 chimeras per group) at E18.5. Mean±s.e FIG. 5B. The organ (liver, lung, intestine, and pancreas) per blood chimerism ratio of mouse Inr knockout chimera (Inr KO, n=4 chimeras per group) or wild-type chimera (WT, n=5 chimeras per group) at E18.5. Mean±s.e. *P<0.05.

### DETAILED DESCRIPTION

Methods of generating functional human organs and tissue in animal bodies suitable for transplantation into human subjects are provided. In particular, the contribution of human donor cells to tissues and organs can be increased in interspecies host embryos by knocking out a growth factor receptor gene such as the insulin-like growth factor 1 receptor or insulin receptor gene. Almost entirely donor-derived functional organs and tissue can be generated by using this method. The methods described herein are useful for generating human organs and tissue in animals and may be helpful for overcoming the current problems with organ shortage for transplantation therapy. Additionally, such organs and tissue can be used in drug discovery, drug screening, and toxicology testing.

Before the chimeric animal donors comprising growth factor gene knockouts and methods of using them to produce chimeric organs for transplantation are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a cell” includes a plurality of such cells and reference to “the embryo” includes reference to one or more embryos and equivalents thereof, e.g., blastocysts or morulas, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as peripheral blood mononuclear cells, fibroblasts, keratinocytes, epithelial cells, endothelial progenitor cells, mesenchymal stem cells, adipose cells, leukocytes, hematopoietic stem cells, bone marrow cells, or hepatocytes.

As used herein, “reprogramming factors” refers to one or more, i.e., a cocktail, of biologically active factors that act on a cell to alter transcription, thereby reprogramming a cell to multipotency or to pluripotency. Reprogramming factors may be provided individually or as a single composition, that is, as a premixed composition, of reprogramming factors to the cells, e.g., somatic cells from an individual with a family history or genetic make-up of interest, such as a patient who has a neurological disorder or a neurodegenerative disease. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

The somatic cells may include, without limitation, peripheral blood mononuclear cells, fibroblasts, keratinocytes, epithelial cells, endothelial progenitor cells, mesenchymal stem cells, adipose cells, leukocytes, hematopoietic stem cells, bone marrow cells, or hepatocytes, etc., which are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector.

By “container” is meant a glass, plastic, or metal vessel that can provide an aseptic environment for culturing cells.

The term “animal” is used herein to include all vertebrate animals, except humans. The term also includes animals at all stages of development, including embryonic, fetal, neonate, and adult stages. Animals may include any member of the subphylum chordata, including, without limitation, non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

As used herein, the term “chimeric organ” or “chimeric tissue” refers to an organ or a tissue comprising cells from different species.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, N.Y., Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells.

A “CRISPR system” refers collectively to transcripts and other elements involved in the expression of or directing the activity of CRISPR-associated (“Cas”) genes. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks).

A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

By “selectively binds” with reference to a guide RNA is meant that the guide RNA binds preferentially to a target sequence of interest or binds with greater affinity to the target sequence than to other genomic sequences. For example, a gRNA will bind to a substantially complementary sequence and not to unrelated sequences. A gRNA that “selectively binds” to a particular allele, such as a particular mutant allele (e.g., allele comprising a substitution, insertion, or deletion), denotes a gRNA that binds preferentially to the particular target allele, but to a lesser extent to a wild-type allele or other sequences. A gRNA that selectively binds to a particular target DNA sequence will selectively direct binding of Cas9 to a substantially complementary sequence at the target site and not to unrelated sequences.

The term “donor polynucleotide” refers to a polynucleotide that provides a sequence of an intended edit to be integrated into the genome at a target locus by homology directed repair (HDR).

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele).

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. The nucleotide sequence comprising the intended edit is integrated into the genomic DNA by HDR or recombineering at the genomic target locus recognized (i.e., sufficiently complementary for hybridization) by the 5′ and 3′ homology arms.

“Administering” a nucleic acid to a cell comprises transducing, transfecting, electroporating, translocating, fusing, phagocytosing, shooting or ballistic methods, etc., i.e., any means by which a nucleic acid can be transported across a cell membrane.

### Chimeric Donor for Use in Transplantation

A chimeric donor is created by genetically modifying a non-human animal host embryo by deleting or inactivating a growth factor receptor gene and transplanting mammalian stem cells having a functional growth factor receptor gene into the non-human animal host embryo. Chimeric organs and tissue comprising mammalian cells are produced from the transplanted mammalian stem cells as the non-human animal host embryo grows. In certain embodiments, the growth factor receptor gene that is deleted or inactivated in the non-human animal host embryo is an insulin-like growth factor 1 receptor (IGF1 R) or an insulin receptor (INR also known as INSR) gene. In certain embodiments, the mammalian stem cells are transplanted into the non-human animal host embryo at the blastula or morula stage. In some embodiments, the stem cells are human stem cells.

The non-human animal can be any non-human animal known in the art that can be used in the methods as described herein. Such animals include, without limitation, non-human primates such as chimpanzees, gorillas, orangutans, and other apes and monkey species, cattle, sheep, pigs, goats, horses, deer, dogs, cats, ferrets, and rodents such as mice, rats, guinea pigs, hamsters, and rabbits.

### Genetically Modifying a Non-Human Animal Embryo to Create a Growth Factor-Deficient Host

The genome of the non-human animal host embryo may be genetically modified to delete or inactivate a growth factor gene (i.e., gene knockout) using standard methods in the art. Typically, the non-human animal host embryo is genetically modified at the zygote stage. In some embodiments, a site-specific nuclease is used to create a DNA break that can be repaired by homology directed repair (HDR) or non-homologous end joining (NHEJ) to produce a knockout of a growth factor receptor gene. In HDR, a donor polynucleotide is used comprising an intended edit sequence to be integrated into the genomic target locus. The donor polynucleotide is used, for example, to delete all or a portion of the growth factor gene or introduce a frameshift mutation. In NHEJ, the two DNA ends at the DNA break, produced by a site-specific nuclease, are ligated together imperfectly, resulting in incorporation of insertions or deletions of base pairs to create a frameshift mutation.

A DNA break may be created by a site-specific nuclease, such as, but not limited to, a Cas nuclease (e.g., Cas9, Cpf1, or C2c1), an engineered RNA-guided Fokl nuclease, a zinc finger nuclease (ZFN), a transcription activator-like effector-based nuclease (TALEN), a restriction endonuclease, a meganuclease, a homing endonuclease, and the like. Any site-specific nuclease that selectively cleaves a sequence at a target site for knockout of a growth factor gene may be used. For a description of genome editing using site-specific nucleases, see, e.g., Targeted Genome Editing Using Site-Specific Nucleases: ZFNs, TALENs, and the CRISPR/Cas9 System (T. Yamamoto ed., Springer, 2015); Genome Editing: The Next Step in Gene Therapy (Advances in Experimental Medicine and Biology, T. Cathomen, M. Hirsch, and M. Porteus eds., Springer, 2016); Aachen Press Genome Editing (CreateSpace Independent Publishing Platform, 2015); herein incorporated by reference.

In some embodiments, genome modification is performed using HDR with a donor polynucleotide comprising a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%), 92%, 93%, 94%, 95%, 96%, 97%, 98%, 99% or 100% sequence identity thereto, wherein the nucleotide sequence comprising the intended edit is integrated into the genomic DNA by HDR at the genomic target locus recognized (i.e., sufficiently complementary for hybridization) by the 5′ and 3′ homology arms.

In certain embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In a preferred embodiment, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g., 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another, e.g., one may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g., one may be 40% shorter or more, 50% shorter or more, sometimes 60% shorter or more, 70% shorter or more, 80% shorter or more, 90% shorter or more, or 95% shorter or more than the other homology arm.

The donor polynucleotide is used in combination with a site-specific nuclease. In some embodiments, the site-specific nuclease is an RNA-guided nuclease, which is targeted to a particular genomic sequence (i.e., genomic target sequence to be modified) by a guide RNA (g RNA). A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to a target sequence in a gene of interest.

In certain embodiments, a CRISPR system is used to knockdown or knockout a growth factor gene in the non-human animal host embryo to produce a chimeric organ or tissue donor. In such embodiments, the RNA-guided nuclease used for genome modification is a CRISPR system Cas nuclease. Any RNA-guided Cas nuclease capable of catalyzing site-directed cleavage of DNA to allow integration of donor polynucleotides by the HDR mechanism can be used in genome editing, including CRISPR system type I, type II, or type III Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, Cas12a (Cpf1), Cas12b (C2c1), Cas12c (C2c3), Cas12d (CasY), Cas12e (CasX), CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquis (NC_018721); Streptococcus thermophilus (YP_820832), Streptococcus mutans (WP_061046374, WP_024786433); Listeria innocua (NP 472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacteriol. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a 5′ protospacer-adjacent motif (PAM) in the DNA at or near the gRNA-binding site.

The genomic target site will typically comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, also known as Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

Cas12b (C2c1) is another class II CRISPR/Cas system RNA-guided nuclease that may be used. C2c1, similarly to Cas9, depends on both a crRNA and tracrRNA for guidance to target sites. For a description of Cas12b, see, e.g., Shmakov et al. (2015) Mol Cell. 60(3):385-397, Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (Fokl-dCas9), wherein the dCas9 portion confers guide RNA-dependent targeting on Fokl. For a description of engineered RNA-guided Fokl nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

An RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector). In some embodiments, the RNA-guided nuclease and the gRNA are both provided by vectors. Both can be expressed by a single vector or separately on different vectors. The vector(s) encoding the RNA-guided nuclease an gRNA may be included in a CRISPR expression system to target a growth factor gene in the non-human animal host embryo.

Codon usage may be optimized to improve production of an RNA-guided nuclease in a particular cell or organism. For example, a nucleic acid encoding an RNA-guided nuclease or reverse transcriptase can be modified to substitute codons having a higher frequency of usage in a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells (e.g., neurons or glia), the protein can be transiently, conditionally, or constitutively expressed in the cell.

In another embodiment, CRISPR interference (CRISPRi) is used to repress gene expression of a growth factor gene in the non-human animal host embryo. CRISPRi is performed with a complex of a catalytically inactive Cas9 (dCas9) with a guide RNA that targets the gene of interest. An engineered nuclease-deactivated Cas9 (dCas9) is used to allow sequence-specific targeting without cleavage. Nuclease-deactivated forms of Cas9 may be engineered by mutating catalytic residues at the active site of Cas9 to destroy nuclease activity. Any such nuclease deficient Cas9 protein from any species may be used as long as the engineered dCas9 retains gRNA-mediated sequence-specific targeting. In particular, the nuclease activity of Cas9 from Streptococcus pyogenes can be deactivated by introducing two mutations (D10A and H841A) in the RuvC1 and HNH nuclease domains. Other engineered dCas9 proteins may be produced by similarly mutating the corresponding residues in other bacterial Cas9 isoforms. For a description of engineered nuclease-deactivated forms of Cas9, see, e.g., Qi et al. (2013) Cell 152:1173-1183, Dominguez et al. (2016) Nat. Rev. Mol. Cell. Biol. 17(1):5-15; herein incorporated by reference in their entireties.

The dCas9 protein can be designed to target a gene of interest by altering its guide RNA sequence. A target-specific single guide RNA (sgRNA) comprises a nucleotide sequence that is complementary to a target site, and thereby mediates binding of the dCas9-sgRNA complex by hybridization at the target site. CRISPRi can be used to sterically repress transcription by blocking either transcriptional initiation or elongation by designing a sgRNA with a sequence complementary to a promoter or exonic sequence. The sgRNA may be complementary to the non-template strand or the template strand, but preferably is complementary to the non-template strand to more strongly repress transcription.

The target site will typically comprise a nucleotide sequence that is complementary to the sgRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide.

In certain embodiments, the sgRNA comprises 5-50 nucleotides, 10-30 nucleotides, 15-25 nucleotides, 18-22 nucleotides, 19-21 nucleotides, and any length between the stated ranges, including, for example, 10, 11, 12, 13, 14, 15, 16, 17, 18, 19, 20, 21, 22, 23, 24, 25, 26, 27, 28, 29, or 30 nucleotides.

The sgRNAs are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109.

In some embodiments, the dCas9 is fused to a transcriptional repressor domain capable of further repressing transcription of the gene of interest, e.g., by inducing heterochromatinization. For example, a Krüppel associated box (KRAB) can be fused to dCas9 to repress transcription of a target gene in human cells (see, e.g., Gilbert et al. (2013) Cell. 154 (2): 442-45, O'Geen et al. (2017) Nucleic Acids Res. 45(17):9901-9916; herein incorporated by reference).

Alternatively, dCas9 can be used to introduce epigenetic changes that reduce expression of a growth factor gene by fusion of dCas9 to an epigenetic modifier such as a chromatin-modifying epigenetic enzyme. The promoter for the gene of interest can be silenced, for example, by methylation or acetylation (e.g., histone H3 lysine 9 [H3K9] methylation, histone H3 lysine 27 [H3K27] methylation, and/or DNA methylation). For example, fusion of dCas9 to a DNA methyltransferase such as DNA methyltransferase 3 alpha (DNMT3A) or a chimeric Dnmt3a/Dnmt3L methyltransferase (DNMT3A3L) allows targeted DNA methylation. Fusion of dCas9 to histone demethylase LSD1 allows targeted histone demethylation (see, e.g., Liu et al. (2016) Cell 167(1):233-247, Lo et al. (2017) F1000Res. 6. pii: F1000 Faculty Rev-747, and Stepper et al. (2017) Nucleic Acids Res. 45(4):1703-1713; herein incorporated by reference).

In yet other embodiments, an RNA-targeting CRISPR-Cas13 system is used to perform RNA interference to reduce expression of a growth factor gene. Members of the Cas13 family are RNA-guided RNases containing two HEPN domains having RNase activity. In particular, Cas13a (C2c2), Cas13b (C2c6), and Cas13d can be used for RNA knockdown. Cas13 proteins can be made to target and cleave transcribed RNA using a gRNA with complementarity to the target transcript sequence. The gRNA is typically about 64 nucleotides in length with a short hairpin crRNA and a 28-30 nucleotide spacer that is complementary to the target site on the RNA transcript. Cas13 recognition and cleavage of a target transcript results in degradation of the transcript as well as nonspecific degradation of any nearby transcripts. See, e.g., Abudayyeh et al. (2017) Nature 550:280-284, Hameed et al. (2019) Microb. Pathog. 133:103551, Wang et al. (2019) Biotechnol Adv. 37(5):708-729, Aman et al. (2018) Viruses 10(12). pii: E732, and Zhang et al. (2018) Cell 175(1):212-223; herein incorporated by reference.

### Stem Cells

The mammalian stem cells can be introduced into the animal host embryo at the blastocyst or morula stage. In some embodiments, transplantation of the mammalian stem cells is performed in utero to a conceptus or to an embryo in in vitro culture. For example, stem cells can be injected into a blastocyst cavity near the inner cell mass or aggregated with morula-stage embryo cells. In some embodiments, at least 5, at least 6, at least 7, at least 8, at least 9, or at least 10 stem cells or more are introduced into the animal host embryo. In some embodiments, 5-10 stem cells are introduced into the animal host embryo, including any number of stem cells within this range such as 5, 6, 7, 8, 9, or 10 stem cells. The mammalian stem cells transplanted into the animal host embryo may be any type of stem cell, including, without limitation, embryonic stem cells, adult stem cells, or induced pluripotent stem cells (IPSCs). The stem cells will generally be of the same species as the mammalian subject receiving the transplant from the chimeric donor. In some embodiments, the mammalian stem cells are human stem cells.

IPSCs can be generated by reprogramming somatic cells into pluripotent stem cells.

Somatic cells can be induced into forming pluripotent stem cells, for example, by treating them with reprograming factors such as Yamanaka factors, including but not limited to, OCT3, OCT4, SOX2, KLF4, c-MYC, NANOG, and LIN28 (see, e.g., Takahashi et al. (2007) Cell. 131(5):861-872; herein incorporated by reference in its entirety). The types of somatic cells that may be converted into IPSCs include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes. Somatic cells are contacted with reprogramming factors in a combination and quantity sufficient to reprogram the cells to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Methods for “introducing a cell reprogramming factor into somatic cells are not limited in particular, and known procedures can be selected and used as appropriate. For example, when a cell reprogramming factor as described above is introduced into somatic cells of the above-mentioned type in the form of proteins, such methods include ones using protein introducing reagents, fusion proteins with protein transfer domains (PTDs), electroporation, and microinjection. When a cell reprogramming factor as described above is introduced into somatic cells of the above-mentioned type in the form of nucleic acids encoding the cell reprogramming factor, a nucleic acid(s), such as cDNA(s), encoding the cell reprogramming factor can be inserted in an appropriate expression vector comprising a promoter that functions in somatic cells, which then can be introduced into somatic cells by procedures such as infection, lipofection, liposomes, electroporation, calcium phosphate coprecipitation, DEAE-dextran, microinjection, and electroporation. Examples of an “expression vector” include viral vectors, such as lentiviruses, retroviruses, adenoviruses, adeno-associated viruses, and herpes viruses; and expression plasmids for animal cells. For example, retroviral or Sendai virus (SeV) vectors are commonly used to introduce a nucleic acid(s) encoding a cell reprogramming factor as described above into somatic cells.

A sample comprising somatic cells is obtained from the subject. The somatic cells may include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes, and other cell types capable of generating patient-derived IPSCs, The biological sample comprising somatic cells is typically whole blood, buffy coat, peripheral blood mononucleated cells (PBMCS), skin, fat, or a biopsy, but can be any sample from bodily fluids, tissue or cells that contain suitable somatic cells. A biological sample can be obtained from a subject by conventional techniques. For example, blood can be obtained by venipuncture, and solid tissue samples can be obtained by surgical techniques according to methods well known in the art.

In some embodiments, the mammalian stem cells that are transplanted into the non-human animal host embryo are adult stem cells. Exemplary adult stem cells include, without limitation, mesenchymal stem cells (e.g., from placenta, adipose tissue, lung, bone marrow, or blood), hematopoietic stem cells, mammary stem cells, intestinal stem cells, endothelial stem cells, and neural stem cells.

The stem cells or somatic cells from which IPSCs are generated are preferably obtained from the mammalian subject that will be receiving the chimeric organ or tissue transplant. Alternatively, the cells can be obtained directly from a donor, a culture of cells from a donor, or from established cell culture lines. Cells are preferably of the same immunological profile as the subject receiving the transplant. Adult stem cells and somatic cells can be obtained, for example, by biopsy from a close relative or matched donor.

The mammalian stem cells express a functional or wild-type growth factor gene (i.e., the growth factor gene that is deficient in the non-human animal host embryo where the growth factor gene is deleted or inactivated). In certain embodiments, the mammalian stem cells are genetically modified to overexpress the growth factor gene. Overexpression of the growth factor can be accomplished, for example, by cloning a nucleic acid encoding the growth factor into an expression vector to create an expression cassette and transfecting the stem cells with the expression vector.

Representative mammalian IGF1R and INR sequences are listed in the National Center for Biotechnology Information (NCBI) database. See, for example, NCBI entries for human IGF1R and INR sequences: Accession Nos. NM_001291858, NM_000875, XM_011521517, XM_017022136, NM_001079817, NM_000208, XM_011527989, XM_011527988, NG 008852; all of which sequences (as entered by the date of filing of this application) are herein incorporated by reference.

Expression cassettes typically include control elements operably linked to the coding sequence, which allow for the expression of the gene in mammalian cells. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. A promoter can be selected that overexpresses the growth factor gene. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems.

Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides encoding the growth factor is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the growth factor include those derived from the pox family of viruses, including vaccinia virus and avian poxvirus. By way of example, vaccinia virus recombinants expressing the growth factor can be constructed as follows. The DNA encoding the growth factor coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides encoding the growth factor. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a growth factor expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Feigner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes of the present invention. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

### Transplantation

Chimeric organs, tissue, and cells produced by the chimeric donor according to the methods described herein can be used for transplantation. In some cases, the chimeric organ is a kidney, a lung, a heart, an intestine, a pancreas, a thymus, a liver, kidney tissue, lung tissue, heart tissue, intestine tissue, pancreas tissue, thymus tissue, liver tissue, or connective tissue. In some cases, the organ may be a complete organ. In other cases, the organ may be a portion of an organ. In other cases, the organ may be cells from a tissue of an organ.

In certain embodiments, at least 70%, at least 75%, at least 80%, at least 85%, at least 90%, or at least 95% or more of the cells in the chimeric organ or tissue are derived from the mammalian stem cells transplanted into the non-human animal host embryo. In some embodiments, 70-100% of the cells in the chimeric organ or tissue are derived from the mammalian stem cells transplanted into the non-human animal host embryo, including any percent within this range, such as 70, 71, 72, 73, 74, 75, 76, 77, 78, 79, 81, 82, 83, 84, 85, 86, 87, 88, 89, 90, 91, 92, 93, 94, 95, 96, 97, 98, 99, or 100%.

Organs, tissue, or cells can be harvested from the chimeric donor and transplanted to a mammalian recipient. Organs, tissue, or cells may be transplanted from the chimeric donor to a recipient such that the organ, tissue, or cells are placed into the appropriate position in the recipient body. In some cases, cardiovascular connections with an organ may be physiologically integrated into the recipient body. The organ, tissue, or cells are preferably from a living chimeric donor but, in some cases, may be from a deceased chimeric donor as long as the organ, tissue, or cells remain viable. The mammalian recipient of the transplant will typically be human. However, the methods described herein may also find use in veterinarian applications such as for treatment of farm animals such as cattle, sheep, pigs, goats and horses and domestic mammals (e.g., pets) such as dogs and cats.

In some cases, an immune response may be mounted against the organ or tissue after transplantation. During such episodes, the transplanted organ or tissue may suffer diminished function or damage. The function and survival of the transplanted organ or tissue may be improved by administration of an immunosuppressive agent. Exemplary immunosuppressive agents include, without limitation, glucocorticoids, such as prednisone, dexamethasone, and hydrocortisone; calcineurin inhibitors such as tacrolimus and ciclosporin; mTOR inhibitors such as sirolimus, everolimus, and zotarolimus; cytostatics such as methotrexate, dactinomycin, anthracyclines, mitomycin C, bleomycin, and mithramycin; and antibodies such as anti-CD20, anti-CD25, and anti-CD3 monoclonal antibodies. Such immunosuppressive agents may be used in treating transplant rejection.

Diagnosis of a rejection episode may utilize clinical data, markers for activation of immune function, markers for tissue damage, and the like. Histological signs include infiltrating T cells, perhaps accompanied by infiltrating eosinophils, plasma cells, and neutrophils, particularly in telltale ratios, structural compromise of tissue anatomy, varying by tissue type transplanted, and injury to blood vessels. Tissue biopsy is restricted, however, by sampling limitations and risks/complications of the invasive procedure. Cellular magnetic resonance imaging (MRI) of immune cells radiolabeled in vivo may provide noninvasive testing.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of creating a chimeric organ or tissue donor, the method comprising:
a) genetically modifying a non-human animal host embryo by deleting or inactivating a growth factor receptor gene; and
b) transplanting mammalian stem cells having a wild-type growth factor receptor gene into the non-human animal host embryo, wherein chimeric organs and tissue comprising mammalian cells are produced from the mammalian stem cells as the non-human animal host embryo grows.

2. The method of aspect 1, wherein the growth factor receptor gene is an insulin-like growth factor 1 receptor (IGF1 R) or an insulin receptor (INR) gene.

3. The method of aspect 1 or 2, wherein the non-human animal is a vertebrate.

4. The method of aspect 3, wherein the vertebrate is a mammal.

5. The method of any one of aspects 1 to 4, wherein the non-human host animal embryo is at the blastocyst stage or morula stage.

6. The method of any one of aspects 1 to 5, wherein the mammalian stem cells are embryonic stem cells, adult stem cells, or induced pluripotent stem cells.

7. The method of any one of aspects 1 to 6, wherein the mammalian stem cells are human stem cells.

8. The method of any one of aspects 1 to 7, wherein the mammalian stem cells are genetically modified to overexpress the growth factor receptor gene.

9. The method of any one of aspects 1 to 8, wherein said transplanting the mammalian stem cells is performed in utero to a conceptus or to the embryo in in vitro culture.

10. The method of any one of aspects 1 to 9, wherein said genetically modifying the non-human animal host embryo comprises using a clustered regularly interspaced short palindromic repeats (CRISPR) system, a transcription activator-like effector nuclease (TALEN), or a zinc-finger nuclease to delete or inactivate the growth factor receptor gene.

11. The method of aspect 10, wherein the CRISPR system, TALEN, or zinc-finger nuclease is used to delete or introduce a frameshift mutation in at least one allele of the growth factor receptor gene.

12. The method of aspect 11, wherein the CRISPR system, TALEN, or zinc-finger nuclease is used to delete or introduce a frameshift mutation in both alleles of the growth factor receptor gene.

13. The method of any one of aspects 10 to 12, wherein the CRISPR system targets an insulin-like growth factor 1 receptor (IGF1 R) or insulin receptor (INR) gene or RNA transcript or makes epigenetic changes that reduce expression of the IGF1 R or the INR gene.

14. The method of aspect 13, wherein the CRISPR system comprises Cas9, Cas12a, Cas12d, Cas13a, Cas13b, Cas13d, or a dead Cas9 (dCas9).

15. The method of aspect 13 or 14, wherein the CRISPR system comprises a single guide RNA (sgRNA) targeting the IGF1 R or the INR gene.

16. A chimeric organ or tissue donor produced by the method of any one of aspects 1 to 15.

17. A method of transplanting an organ or tissue into a mammalian recipient subject, the method comprising transplanting a chimeric organ or tissue from the chimeric organ or tissue donor of aspect 16 to the mammalian recipient subject.

18. The method of aspect 17, wherein at least 90% of the cells in the chimeric organ or tissue are produced from the mammalian stem cells.

19. The method of aspect 18, wherein the stem cells are human stem cells.

20. The method of any one of aspects 17 to 19, wherein the mammalian stem cells are adult stem cells from the mammalian recipient subject.

21. The method of any one of aspects 17 to 19, wherein the mammalian stem cells are induced pluripotent stem cells derived from cells from the mammalian recipient subject.

22. The method of any one of aspects 17 to 21, wherein the mammalian recipient subject is human.

23. The method of any one of aspects 17 to 22, wherein the chimeric organ or tissue is a kidney, a lung, a heart, an intestine, a pancreas, a thymus, a liver, kidney tissue, lung tissue, heart tissue, intestine tissue, pancreas tissue, thymus tissue, liver tissue, or connective tissue.

24. The method of any one of aspects 17 to 23, further comprising administering an immunosuppressive agent to the mammalian recipient subject.

25. A non-human animal host embryo comprising:
a) a genetically modified genome comprising a knockout of an insulin-like growth factor 1 receptor (IGF1R) gene or an insulin receptor (INR) gene; and
b) transplanted mammalian stem cells having a wild-type growth factor receptor gene, wherein said non-human animal host embryo produces chimeric organs and tissue comprising mammalian cells from the mammalian stem cells during development.

26. The non-human animal host embryo of aspect 25, wherein the non-human animal host embryo is a vertebrate.

27. The non-human animal host embryo of aspect 26, wherein the vertebrate is a mammal.

28. The non-human animal host embryo of any one of aspects 25 to 27, wherein the non-human animal host embryo is at the blastocyst stage or morula stage.

29. The non-human animal host embryo of any one of aspects 25 to 28, wherein the mammalian stem cells are embryonic stem cells, adult stem cells, or induced pluripotent stem cells.

30. The non-human animal host embryo of any one of aspects 25 to 28, wherein the mammalian stem cells are human stem cells.

31. The non-human animal host embryo of any one of aspects 25 to 30, wherein the mammalian stem cells are genetically modified to overexpress the IGF1R gene or the INR gene.

32. The non-human animal host embryo of any one of aspects 25 to 31, wherein the knockout comprises a deletion of the IGF1R gene or the INR gene or a frameshift mutation in the IGF1R gene or the INR gene.

33. The non-human animal host embryo of any one of aspects 25 to 32, wherein both alleles of the IGF1R gene or the INR gene are knocked out in the non-human animal host embryo.

34. Use of the non-human animal host embryo of any one of aspects 25 to 33 in the manufacture of a chimeric mammalian organ or tissue.

35. The use of aspect 34, wherein at least 90% of the cells in the chimeric organ or tissue are produced from the mammalian stem cells.

36. The use of aspect 35, wherein the mammalian stem cells are human stem cells.

37. A method of transplanting an organ or tissue into a mammalian subject, the method comprising transplanting a chimeric organ or tissue produced from the non-human animal host embryo of any one of aspects 25 to 33 to the mammalian subject.

38. The method of aspect 37, wherein at least 90% of the cells in the chimeric organ or tissue are produced from the mammalian stem cells.

39. The method of aspect 38, wherein the mammalian stem cells are human stem cells.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Generation of Functional Organs Using a Cell-Competitive Niche in Intra- and Inter-Species Rodent Chimeras

The generation of functional organs using stem cell technology can solve the critical problem of shortages of organs needed for transplantation. Despite advances in deriving tissue-specific cells (De Luca et al., 2019) and organoids (Takebe and Wells, 2019), reconstituting a whole organ in a dish continues to be challenging. We have used blastocyst complementation in rodents to generate fully functional xenogenic organs from pluripotent stem cells (PSCs) in vivo by exploiting developmental organ niches (Goto et al., 2019; Isotani et al., 2011; Kobayashi et al., 2010; Yamaguchi et al., 2017). Nonetheless, a human organ has not yet been generated within a developing animal via blastocyst complementation. A major reason is that very few human cells engraft and contribute to animal tissues (Wu et al., 2017), leaving not enough cells to complement a developmental niche in interspecies chimeras (Goto et al., 2019). Increasing donor cell contribution may substantially benefit efforts to generate functional human organs for clinical transplantation.

Generating an interspecies embryo with robust levels of chimerism encounters both regional and temporal barriers. Xenogenic cells may contribute unevenly to varying lineages, resulting in regions with low chimerism (Yamaguchi et al., 2018). Blastocyst complementation with more donor cells, however, does not boost overall chimerism. High xenogenic cell contribution at early developmental stages is associated with anomalies or embryonic death (Yamaguchi et al., 2018). As this effect is less pronounced between closely related rodent species than between distantly related ones, high chimerism early in development is thought more likely to be lethal between more evolutionarily diverse species. Thus, lower chimerism is advantageous during early development, while higher chimerism may be needed later to complement an organ niche effectively.

Insulin-like growth factor 1 (Igf1) in pre- and post-natal growth in mammals is a key mediator of growth (Baker et al., 1993; Liu et al., 1993; Lupu et al., 2001). It acts through the Igf1 receptor (Igf1r), which is ubiquitously expressed in tissues, modulating mitogenic, anti-apoptotic, and transformational pathways (Bentov and Werner, 2013). The disruption of Igf1 r in mouse embryos induces growth retardation, usually with neonatal death (Baker et al., 1993; Liu et al., 1993). Here, we demonstrate that the deletion of Igf1r in mouse host embryos creates a “cell-competitive niche” that substantially increases donor chimerism in both intra- and interspecies rodent chimeras. Of importance is that Igf1r deletion opens this niche in stages of development later than those affected by the early developmental arrest seen in interspecies highly chimeric fetuses. Donor cells that persist until the niche opens can thereafter proliferate within it. Our observations thus may facilitate in vivo organ generation within interspecies chimeras. Access to an amenable host niche may promote the contribution of donor cells during fetal development.

### Results

### Donor Cells Predominantly Proliferate in Igf1r Null Mouse Embryos Generated Using the CRISPR/Cas9

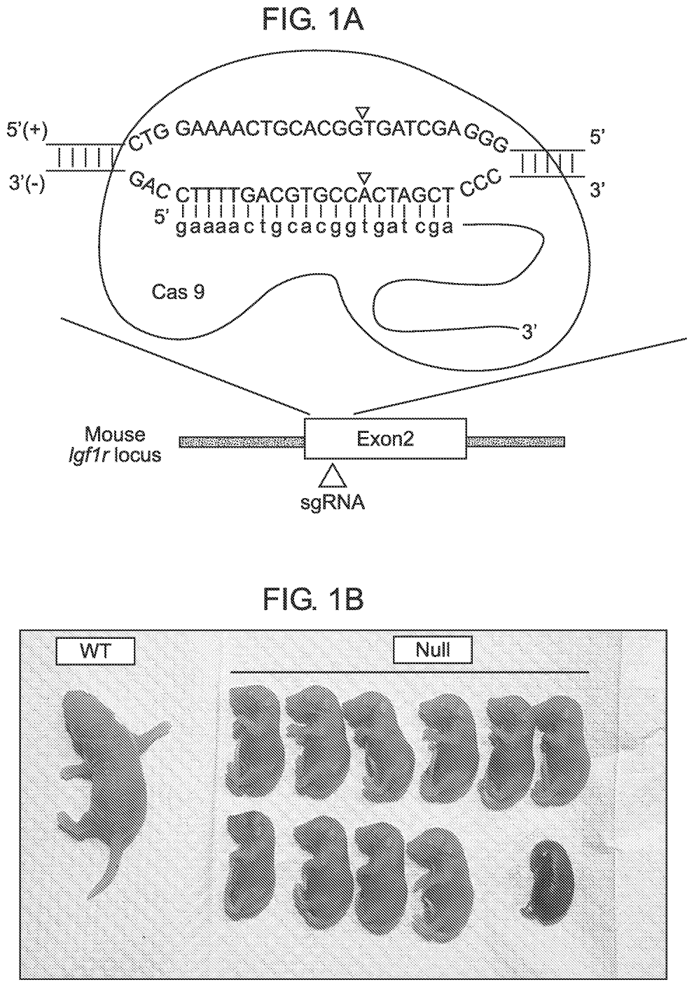

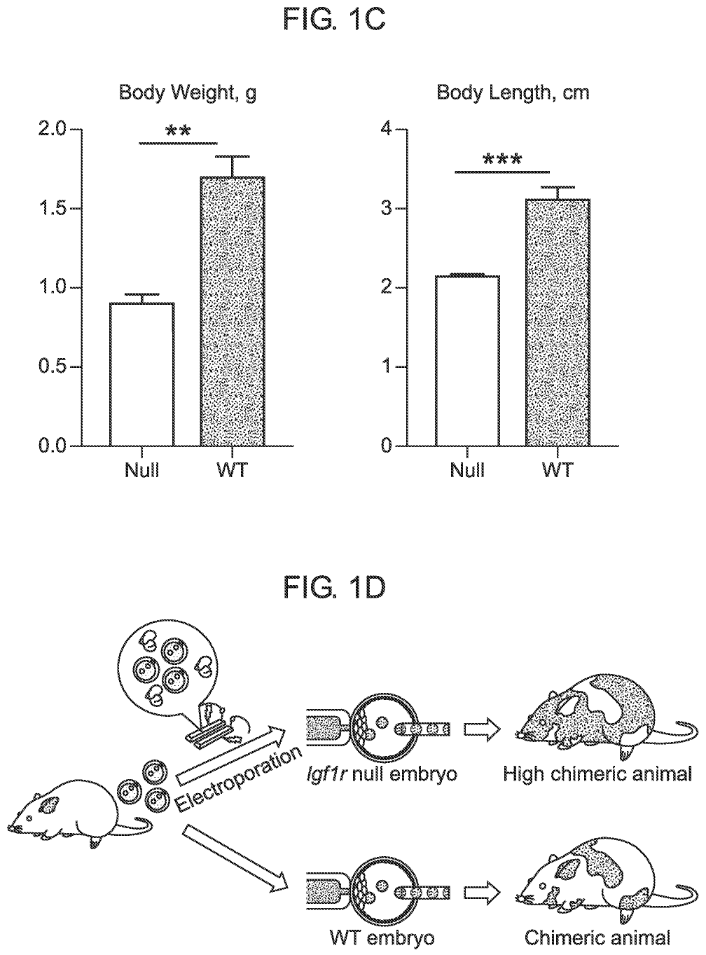

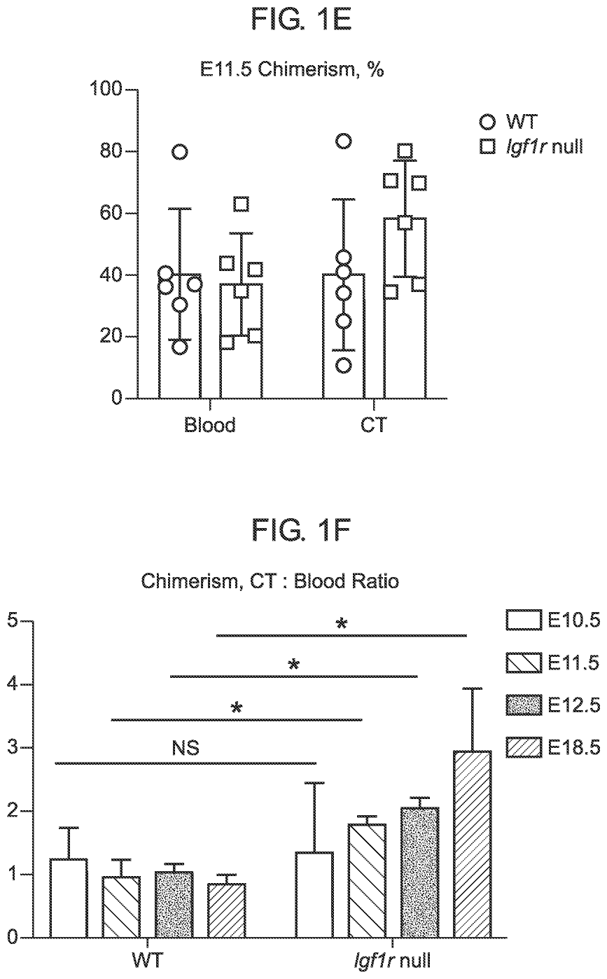

The clustered regularly interspaced short palindromic repeats (CRISPR)-associated protein 9 (CRISPR/Cas9) and single guide RNA (sgRNA) complex targeting the Igf1r gene locus was electroporated into mouse zygotes to induce out-of-frame mutations that lead to the premature termination of transcription (FIG. 1A). Genomic DNA was extracted from blastocysts for Igf1r locus analysis tracking indels by decomposition (TIDE webtool; Brinkman et al., 2014).

As knockout efficiency was higher with sgRNA1 than sgRNA2 (Table 1), sgRNA1 was used in subsequent experiments. Igf1r null neonates were smaller than wild-type (WT) neonates and died postnatally with breathing difficulties, as reported (Baker et al., 1993; Liu et al., 1993; Powell-Braxton et al., 1993) (FIGS. 1B, 1C). IGF1R was not detected in the skin, heart, or bone of Igf1r null neonates.

The behavior of Igf1 in intraspecies chimeric embryos is unexplored. We hypothesized that in embryos composed of WT and Igf1r null cells, WT cells would predominantly proliferate since only they can receive the Igf1 signal. To generate chimeric embryos, we derived a mouse embryonic stem cell (mESC) line with constitutive GFP expression and validated its pluripotency by using teratoma- and chimera-formation assays. The mESCs were then injected into WT and Igf1r null embryos (FIG. 1D). Since Igf1r null mouse embryos do not manifest growth retardation until after embryonic day (E) 10.5 (Baker et al., 1993; Liu et al., 1993), we investigated the chimeric embryo at E11.5. Blood and connective tissue (CT) were collected at E11.5, and chimerism was analyzed by flow cytometry (FIG. 1E). Average CT chimerism increased by nearly 20% in the Igf1r null embryos, while blood chimerism was not affected. As expected, the statistical analysis of chimeras was impeded by a huge variation in systemic chimerism among individual embryos (e.g., WT chimerism varied from ˜10% to ˜90% within a single injection experiment). Since blood chimerism appeared unaffected by knocking out Igf1r, we instead analyzed the ratio of CT:blood chimerism over time. Remarkably, the ratio increased until it was ˜3 times higher at E18.5 only in Igf1r null fetuses (FIG. 1F). In contrast, the CT:blood chimerism ratio was constant in WT embryos throughout and in Igf1r null embryos before E11.5. Analysis of variance (ANOVA) further evaluated the effect of Igf1r knockout with respect to time and CT:blood chimerism. The results indicated that (1) Igf1r knockout resulted in significantly higher CT:blood ratios overall (p=0.001) and (2) the effect of Igf1r knockout was a function of time, increasing with longer embryonic development (p=0.013). Further statistical analysis, using non-parametric testing to accommodate the small sample sizes, demonstrated significance (FIG. 1F). These data suggest that (1) WT donor cells have a growth advantage over Igf1r null host cells in the developing embryo, (2) the extent of growth advantage varies from organ to organ, and (3) it becomes evident after E10.5, regardless of initial chimerism level.

Increasing Organ- and Tissue-Specific Chimerism in Igf1r Null Chimeras during Fetal and Neonatal Mouse Development

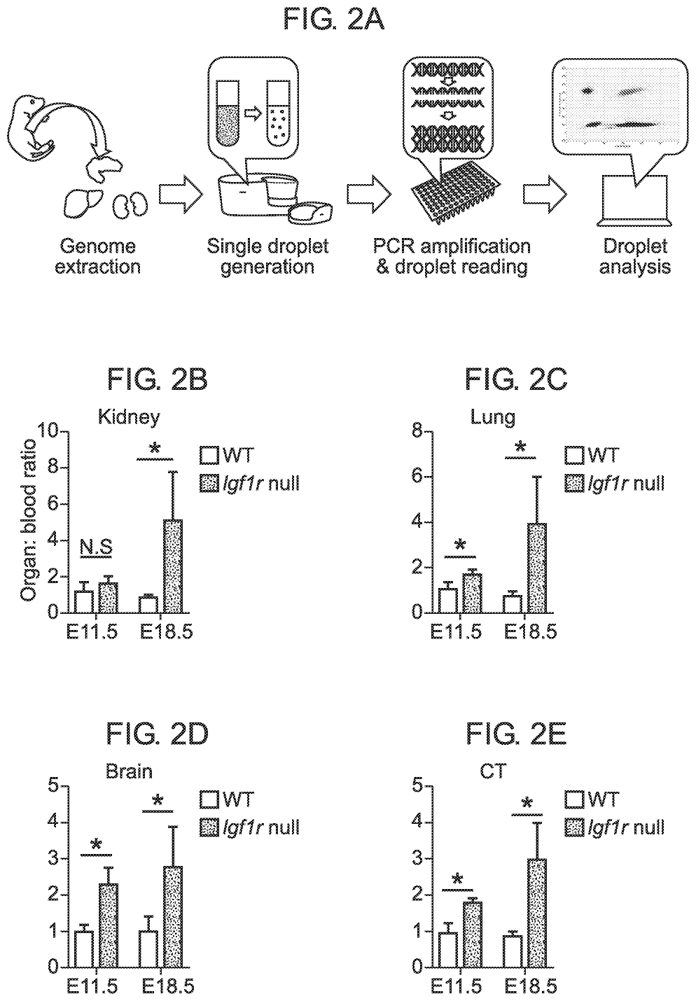

We next examined the extent of donor chimerism in individual tissues of Igf1r null chimeras. Most mouse organs (lung, liver, heart, brain, intestine, kidney, blood, gonad, and thymus) can be identified by appearance on dissecting microscopy from E11.5 onward. We harvested each of these organs or tissues from both Igf1r null and WT chimeras at E11.5 and E18.5 (neonate) and extracted DNA individually from each. Chimerism was analyzed with the droplet digital PCR (ddPCR) platform (FIG. 2A), because it does not require careful single-cell dissociation, which may introduce biases toward specific cell types, and it is unaffected by reporter silencing (Calvo et al., 2020; Hamanaka et al., 2018). The ratio of organ:blood chimerism in Igf1r null chimeras was significantly higher at E11.5 in 7 of 9 organs/tissues (lung, brain, CT, heart, intestine, thymus, and liver) than in WT chimeras and was higher still at E18.5 in almost all organs except the thymus, probably due to the homing of blood cells to the thymus at later developmental stages (Owen and Ritter, 1969) (FIGS. 2B-2E). In addition, at E18.5 gonad:blood chimerism was higher in Igf1r null chimeras than in WT chimeras. The kidney displayed the highest increase in chimerism, with 3- to 10-fold higher levels than that of blood (Table S2). This is consistent with a crucial role for Igf1r in renal development (Rogers et al., 1991, 1999; Wada et al., 1993). Interestingly, Igf1r null chimeras were normal in gross appearance and proportions of body parts, indicating that host and donor tissues grew in concert during development (FIG. 2F). The increase in chimerism thus was not simply due to the unregulated overgrowth of donor cells. These results show that WT cells outcompete Igf1r null cells in developing embryos, although the extent of chimerism varies among organs.

The Cell-Competitive Niche Induces Almost Entirely Donor-Derived Organs in Igf1r Null Mouse Chimeras

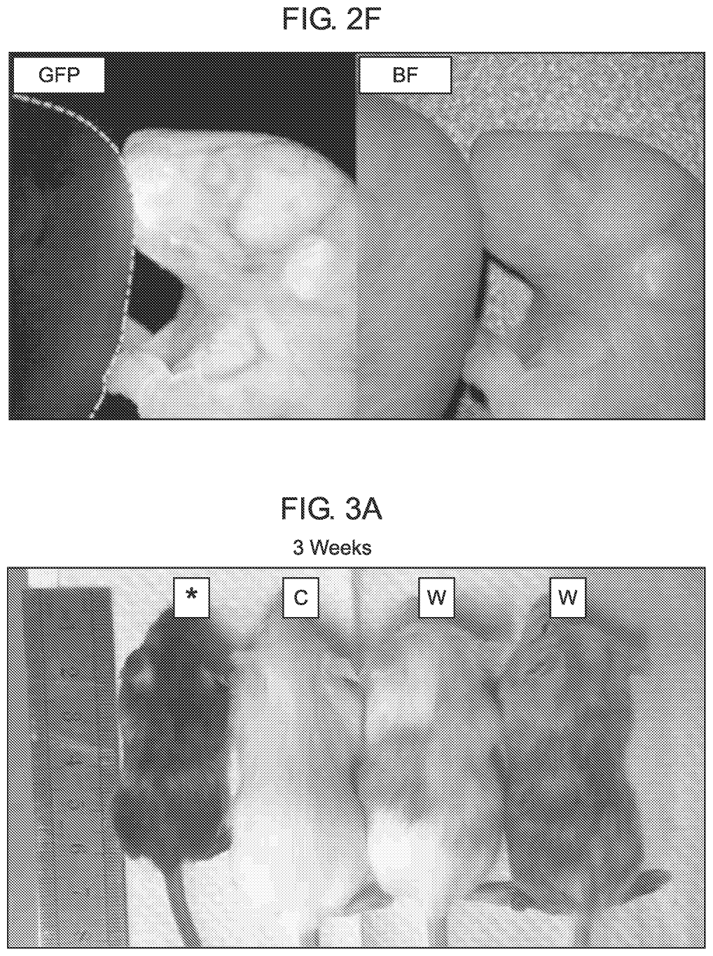

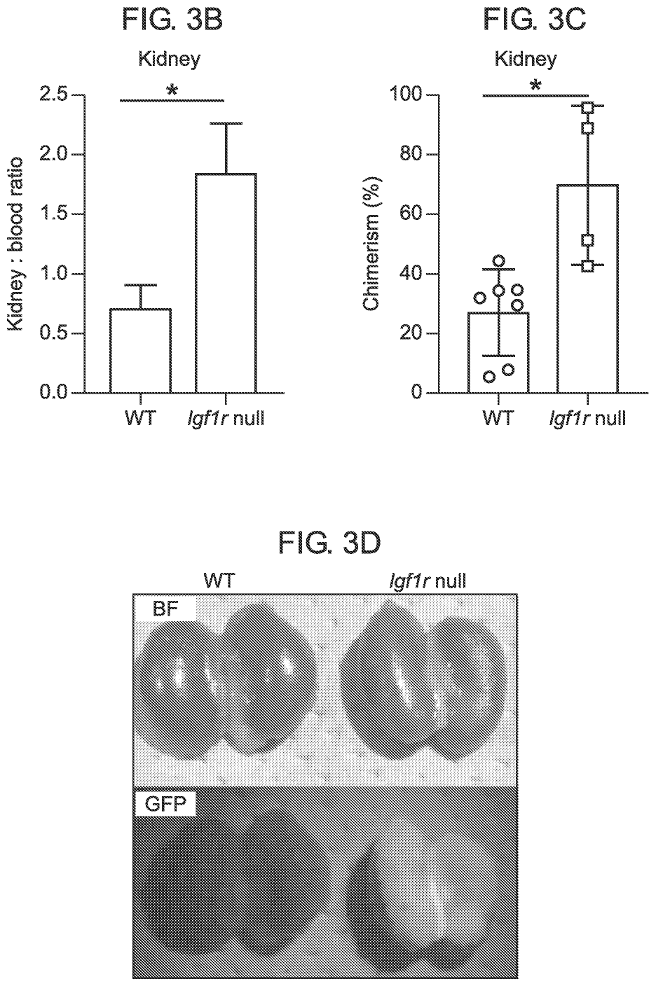

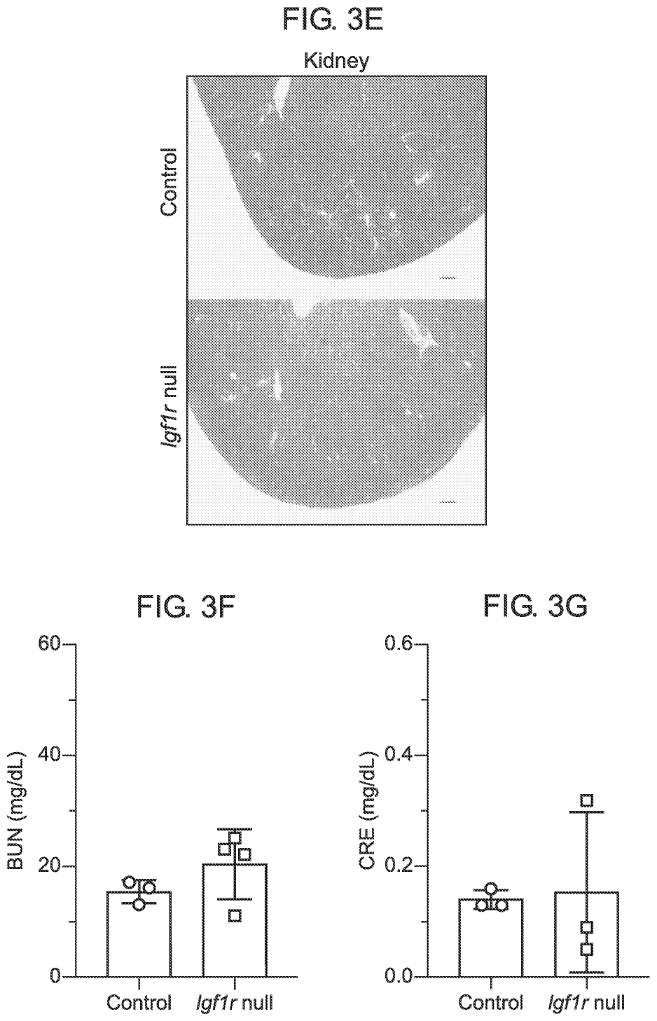

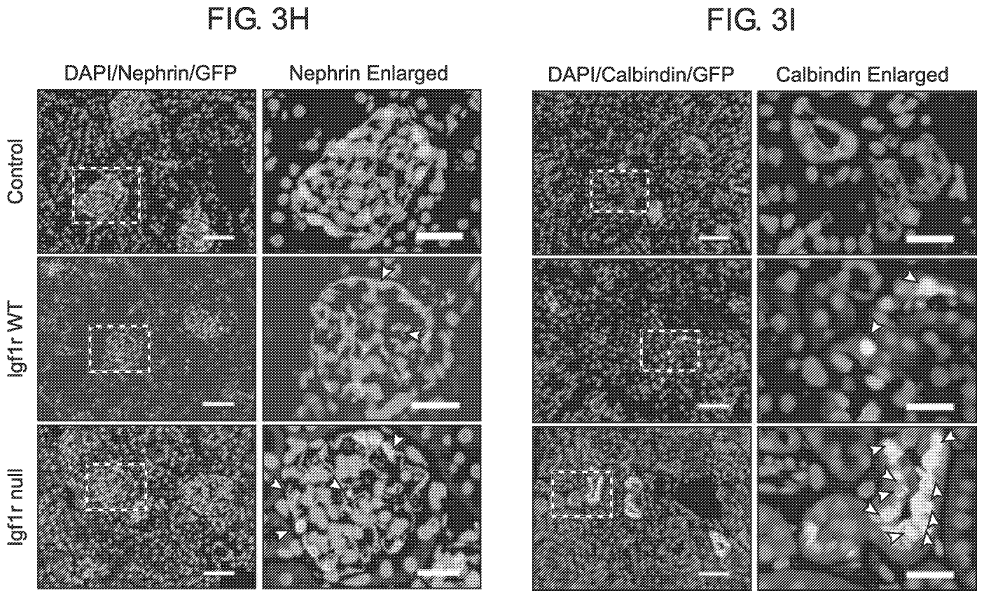

To analyze the postnatal effect of Igf1r disruption, we investigated chimerism in adult Igf1r null chimeras. Although Igf1r disruption causes postnatal death in breathing difficulties, Igf1r null chimeras survived postnatally and grew normally to adulthood (FIG. 3A). This indicates that blastocyst-injected WT cells can rescue chimeras from the lethal phenotype of Igf1r disruption. We again analyzed chimerism in each organ or tissue and normalized it against chimerism in blood (organ:blood chimerism ratio). Kidney, liver, brain, and lung ratios were significantly higher in Igf1r null chimeras than in WT chimeras (FIG. 3B). Organ:blood chimerism ratios in the gonads of adult mice did not shift with genotype, in contrast to the increase in Igf1r null chimerism ratio over the WT chimerism ratio observed in the neonatal gonad. In several Igf1r null mice, absolute chimerism in kidney, brain, and lung approached 100%, which is not the case in other organs or tissues (FIG. 3C). Since chimerism was consistently higher in Igf1r null kidneys than in other Igf1r null organs, we further investigated the structure and function of these almost entirely donor-derived organs. The donor-derived kidneys expressed GFP throughout (FIG. 3D) and were normal on macroscopy and microscopy, without hydronephrosis or fibrosis (FIG. 3E). Other highly chimeric organs in the Igf1r null chimeras were morphologically normal, with unremarkable tissue architecture. Renal function was assessed by measuring serum blood urea nitrogen, creatinine, and albumin concentrations. All of the levels were within normal ranges in both Igf1r null and WT chimeras (FIGS. 3F, 3G). These results indicate that Igf1r null chimeras have high donor cell contributions and can develop into healthy adults. In addition, Igf1r disruption creates a niche that allows donor cells to constitute some organs almost entirely, while maintaining normal structure and function. Immunohistologic techniques were used to identify the extent and distribution of chimerism in the kidneys. Antibodies specific to renal components (nephrin, aquaporin 1, Na⁺/K⁺ ATPase a-1, and calbindin) were used for analysis (Kesti^{€}a et al., 1998; Nielsen et al., 1993; Rhoten et al., 1985; Sabolic et al., 1999). The kidneys of WT chimeras contained a mixture of host and donor cells in all components (FIGS. 3H, 3I). In contrast, the kidneys of Igf1r null chimeras were almost entirely composed of GFP-expressing donor cells, including calbindin-expressing collecting ducts (FIGS. 3H, 3I). Consistent with this result, all of the components of the lung were also almost entirely derived from GFP-expressing donor cells. These results suggest that donor cells can generate and constitute all renal and pulmonary components in the Igf1r null environment.

### Reducing Competition in the Tissue Niche Enhances Donor Cell Contribution in Interspecies Mouse-Rat Chimeras

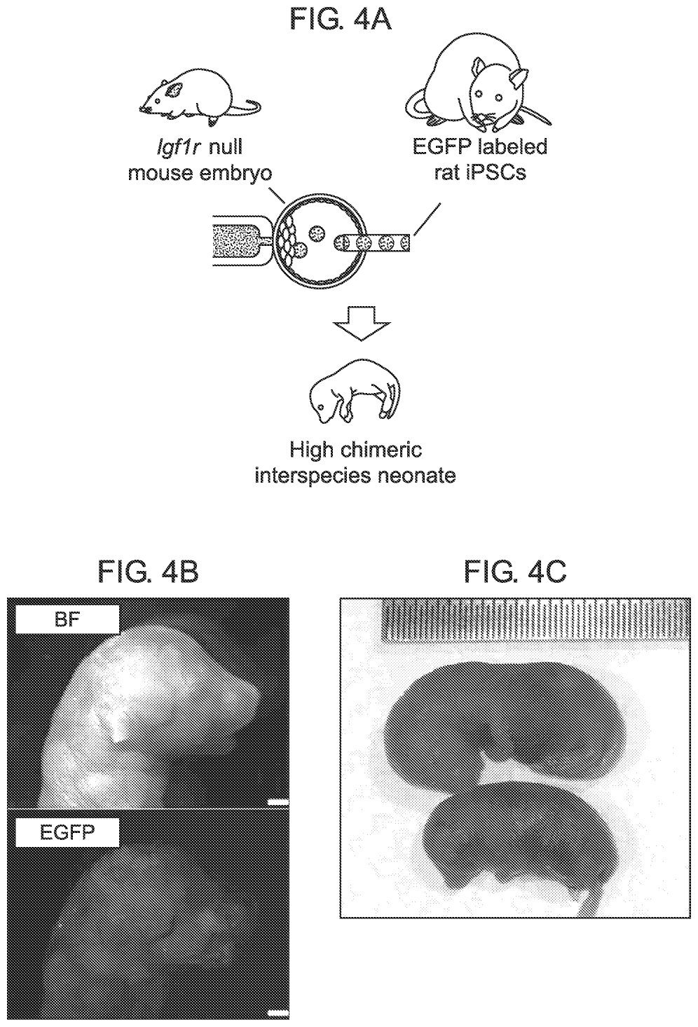

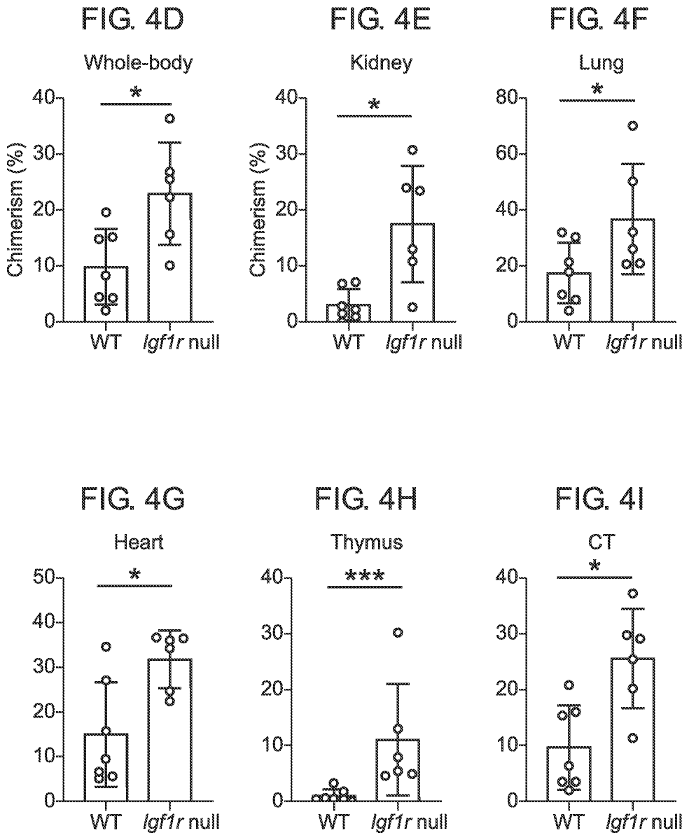

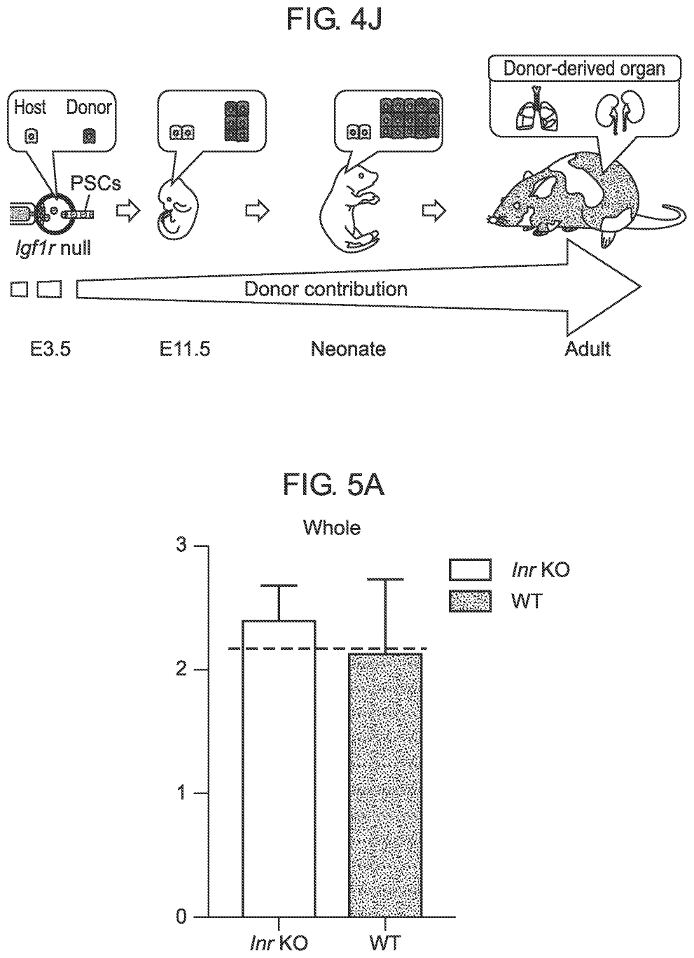

Having established that the Igf1r null host provides a niche that enables WT cells to proliferate predominantly intraspecies, we assessed whether this niche accepted donor cells in an interspecies environment. We injected EGFP-labeled rat induced PSCs (iPSCs; Yamaguchi et al., 2018) into Igf1r null mouse embryos and obtained interspecies chimeric neonates that expressed EGFP (FIGS. 4A and 4B). These interspecies chimeras were larger than Igf1r null mouse neonates (FIG. 4C). Rat PSCs contribute less than mouse PSCs to mouse embryos after blastocyst injection; this is ascribed to an interspecies developmental barrier at ˜E10.5 (Yamaguchi et al., 2018). However, overall rat chimerism increased in Igf1r null chimeras at E18.5, viz., in neonates (FIG. 4D). Donor contribution differs across organs/tissues specifically in interspecies chimeras, and contributions differ from those seen in intraspecies chimeras (Yamaguchi et al., 2018). Thus, for interspecies chimeras, the absolute chimerism observed in each organ was used for subsequent analysis. Rat chimerism in individual organs was significantly higher in the kidney, lung, heart, thymus, and CT (FIGS. 4E-41), but not in the brain, gonad, liver, and intestine. In these organs, Igf1r-mediated signaling plays little or no role in organ development. The liver was particularly free from donor cell contribution in both WT and Igf1r null chimeras. This may suggest a lack of cross-reactivity in the ligand-receptor system required for liver development. Donor chimerism reached almost 70% in the lung in rat-mouse chimeras (FIG. 4F). In addition, the frequency of successful chimeric-fetus generation did not differ between WT and Igf1r null chimeras, suggesting that high chimeric fetuses that were generated using the cell-competitive niche were not affected by early developmental arrest. These results indicate that WT rat cells can multiply to become the dominant population in Igf1r null interspecies animals despite the interspecies barrier. We infer that Igf1r deletion in host embryos selectively favors WT donor cells in both intra- and interspecies chimeras.

### Discussion

Our work demonstrates a proof-of-concept approach for facilitating in vivo organ generation. By opening a competitive cell niche, we could (1) gradually increase donor cell contribution in later stages of development in intra- and interspecies chimeric embryos, (2) evade early developmental arrest observed in inter-species chimeras by increasing systemic chimerism at later developmental stages, and (3) generate entire donor-derived organs in intraspecies chimeras by supplanting host cells within an organ niche (summarized in FIG. 4J).

A barrier prevents a highly chimeric interspecies embryo from developing. This barrier exists in the early developmental stages and impedes in vivo organ generation, since donor-cell contribution must meet a certain level for successful blastocyst complementation (Goto et al., 2019; Yamaguchi et al., 2018). In contrast, opening the cell-competitive niche allows interspecies donor chimerism to increase gradually from mid- to late developmental stages, circumventing the problems of early developmental arrest associated with high donor-cell chimerism during embryogenesis. Igf1r null embryos thus can be used to overcome temporal aspects of the xenogenic developmental barrier.

Variation in chimerism among organs was greater in interspecies chimeras than in intraspecies chimeras. This is common in interspecies environments. To speculate on the role of varying affinities of xenogenic molecules in orthologous signaling pathways is tempting. While mouse PSCs can generate kidneys in a rat, it is much more difficult for rat PSCs to generate kidneys in a mouse (Goto et al., 2019; Usui et al., 2012). Opening the cell-competitive niche increased donor contributions in almost all organs, including those previously found to be challenging. This system thus could overcome the interspecies organ-/tissue-specific barrier.

While a mouse PSC-derived kidney can be generated in a Sall1 null rat, the regenerated kidney contains rat cells in the collecting ducts, suggesting that to knock out a different gene or several genes may be necessary for fully donor-derived kidney generation (Goto et al., 2019). Injected donor PSCs in an Igf1r null host fetus by contrast proliferated and gave rise to the entire kidney, including the collecting ducts. We infer that Igf1r plays crucial roles in whole-kidney development in the mouse.

Opening the cell competitive niche also dramatically increases donor chimerism in interspecies chimeras, despite the less-fit xenogenic environment such as that experienced by rat PSCs in mouse kidneys (Yamaguchi et al., 2018). Donor chimerism in lungs was the highest, reaching almost 70% in rat-mouse chimeric neonates. If these interspecies Igf1r null chimeras had survived to adulthood, then chimerism may well have been much higher. However, all of the interspecies Igf1r null chimeras died perinatally. Those that were liveborn evidenced breathing difficulties that we ascribe to lung problems. To avoid this, we plan an organ-specific knockout of Igf1r that may let the mice grow to adulthood. After birth, the contribution of rat cells should increase further in the target organ. We anticipate that various modulations will permit the opening of the competitive-cell niche to facilitate whole-organ regeneration, even in species more evolutionarily divergent than rats are from mice.

Although several approaches exploit developmental niches (e.g., blastocyst complementation, in utero transplantation) to achieve in vivo organ generation (Ma et al., 2018; Yamanaka et al., 2019), blastocyst complementation has succeeded in generating whole organs from PSCs in vivo only when an empty organ niche has been created in a developing host animal (Goto et al., 2019; Hamanaka et al., 2018; Kobayashi et al., 2010; Yamaguchi et al., 2017). To open the cell-competitive niche differs in that it provides an environment in which donor cells gradually supplant host cells within a developing and growing organ, leading to complete donor derivation. Of relevance is that Igf1 is widely conserved among mammals, including human Igf1 (Rotwein, 2017). Thus, we believe that the strategy that we describe can move forward the generation of human organs in evolutionarily divergent interspecies organ niches.

Our data evince that host-cell lack of Igf1r expression confers selective advantages upon donor cells in most, if not all, organs and tissues. This may result from decreased proliferation or inefficient differentiation of host cells due to the absence of Igf1-mediated signaling, which may be organ specific, but that has not been established. Patterns of Igf1r expression in organs of the postnatal mouse may assist in evaluating this possibility. In rat-mouse inter-species chimeras, selective advantage is less robust in most organs than in mouse-rat interspecies chimeras. This may indicate lower affinity of mouse Igf1 for rat Igf1 r than that of rat Igf1 for mouse Igf1r. Interspecies chimeras promise a better understanding of the cues and pathways required for organogenesis.

In conclusion, these observations will advance our present understanding of cell-cell interaction in fetal development, as well as facilitate interspecies in vivo organ generation. This has direct applications for modeling diseases, exploring developmental biology, and ultimately, generating human organs for transplantation.

### REFERENCES

Baker, J., Liu, J. P., Robertson, E. J., and Efstratiadis, A. (1993). Role of insulin-like growth factors in embryonic and postnatal growth. Cell 75, 73-82.
Bentov, I., and Werner, H. (2013). Insulin-like Growth Factor-1. In Handbook of Biologically Active Peptides, Second Edition, A. J. Kastin, ed. (Academic Press), pp. 1627-1632.
Brinkman, E. K., Chen, T., Amendola, M., and van Steensel, B. (2014). Easy quantitative assessment of genome editing by sequence trace decomposition. Nucleic Acids Res. 42, e168.
Brownstein, D. G. (2003). Manipulating the Mouse Embryo: A Laboratory Manual, Third Edition (University of Chicago Press).
Calvo, B., Rubio, F., Fernández, M., and Tranque, P. (2020). Dissociation of neonatal and adult mice brain for simultaneous analysis of microglia, astrocytes and infiltrating lymphocytes by flow cytometry. IBRO Rep. 8, 36-47.
De Luca, M., Aiuti, A., Cossu, G., Parmar, M., Pellegrini, G., and Robey, P. G. (2019). Advances in stem cell research and therapeutic development. Nat. Cell Biol. 21, 801-811.
Goto, T., Hara, H., Sanbo, M., Masaki, H., Sato, H., Yamaguchi, T., Hochi, S., Kobayashi, T., Nakauchi, H., and Hirabayashi, M. (2019). Generation of pluripotent stem cell-derived mouse kidneys in Sall1-targeted anephric rats. Nat. Commun. 10, 451.
Hamanaka, S., Umino, A., Sato, H., Hayama, T., Yanagida, A., Mizuno, N., Kobayashi, T., Kasai, M., Suchy, F. P., Yamazaki, S., et al. (2018). Generation of vascular endothelial cells and hematopoietic cells by blastocyst complementation. Stem Cell Reports 11, 988-997.
Hashimoto, M., Yamashita, Y., and Takemoto, T. (2016). Electroporation of Cas9 protein/sgRNA into early pronuclear zygotes generates non-mosaic mutants in the mouse. Dev. Biol. 418, 1-9.
Hindson, B. J., Ness, K. D., Masquelier, D. A., Belgrader, P., Heredia, N. J., Makarewicz, A. J., Bright, I. J., Lucero, M. Y., Hiddessen, A. L., Legler, T. C., et al. (2011). High-throughput droplet digital PCR system for absolute quantitation of DNA copy number. Anal. Chem. 83, 8604-8610.
Isotani, A., Hatayama, H., Kaseda, K., Ikawa, M., and Okabe, M. (2011). Formation of a thymus from rat ES cells in xenogeneic nude mouse4rat ES chimeras. Genes Cells 16, 397-405.
Kestila, M., Lenkkeri, U., M€annikkö, M., Lamerdin, J., McCready, P., Putaala, H., Ruotsalainen, V., Morita, T., Nissinen, M., Herva, R., et al. (1998). Positionally cloned gene for a novel glomerular protein—nephrin—is mutated in congenital nephrotic syndrome. Mol. Cell 1, 575-582.
Kobayashi, T., Yamaguchi, T., Hamanaka, S., Katoltoh, M., Yamazaki, Y., Ibata, M., Sato, H., Lee, Y. S., Usui, J., Knisely, A. S., et al. (2010). Generation of rat pancreas in mouse by interspecific blastocyst injection of pluripotent stem cells. Cell 142, 787-799.
Liu, J. P., Baker, J., Perkins, A. S., Robertson, E. J., and Efstratiadis, A. (1993). Mice carrying null mutations of the genes encoding insulin-like growth factor I (Igf-1) and type 1 IGF receptor (Igf1r). Cell 75, 59-72.
Lupu, F., Terwilliger, J. D., Lee, K., Segre, G. V., and Efstratiadis, A. (2001). Roles of growth hormone and insulin-like growth factor 1 in mouse postnatal growth. Dev. Biol. 229, 141-162.
Ma, H., Wert, K. J., Shvartsman, D., Melton, D. A., and Jaenisch, R. (2018). Establishment of human pluripotent stem cell-derived pancreatic b-like cells in the mouse pancreas. Proc. Natl. Acad. Sci. USA 115, 3924-3929.
Mizuno, N., Mizutani, E., Sato, H., Kasai, M., Ogawa, A., Suchy, F., Yamaguchi, T., and Nakauchi, H. (2018). Intra-embryo gene cassette knockin by CRISPR/Cas9-mediated genome editing with adeno-associated viral vector. iScience 9, 286-297.
Mori, M., Furuhashi, K., Danielsson, J. A., Hirata, Y., Kakiuchi, M., Lin, C. S., Ohta, M., Riccio, P., Takahashi, Y., Xu, X., et al. (2019). Generation of functional lungs via conditional blastocyst complementation using pluripotent stem cells. Nat. Med. 25, 1691-1698.
Nielsen, S., Smith, B. L., Christensen, E. I., Knepper, M. A., and Agre, P. (1993). CHIP28 water channels are localized in constitutively water-permeable segments of the nephron. J. Cell Biol. 120, 371-383.
Owen, J. J., and Ritter, M. A. (1969). Tissue interaction in the development of thymus lymphocytes. J. Exp. Med. 129, 431-442.
Powell-Braxton, L., Hollingshead, P., Warburton, C., Dowd, M., Pitts-Meek, S., Dalton, D., Gillett, N., and Stewart, T. A. (1993). IGF-I is required for normal embryonic growth in mice. Genes Dev. 7 (12B), 2609-2617.
Rhoten, W. B., Bruns, M. E., and Christakos, S. (1985). Presence and localization of two vitamin D-dependent calcium binding proteins in kidneys of higher vertebrates. Endocrinology 117, 674-683.
Rogers, S. A., Ryan, G., and Hammerman, M. R. (1991). Insulin-like growth factors I and II are produced in the metanephros and are required for growth and development in vitro. J. Cell Biol. 113, 1447-1453.
Rogers, S. A., Powell-Braxton, L., and Hammerman, M. R. (1999). Insulin-like growth factor I regulates renal development in rodents. Dev. Genet. 24, 293-298.
Rotwein, P. (2017). Diversification of the insulin-like growth factor 1 gene in mammals. PLOS ONE 12, e0189642.
Sabolic, I., Herak-Kramberger, C. M., Breton, S., and Brown, D. (1999). Na/K-ATPase in intercalated cells along the rat nephron revealed by antigen retrieval. J. Am. Soc. Nephrol. 10, 913-922.
Shimizu, T., Ueda, J., Ho, J. C., Iwasaki, K., Poellinger, L., Harada, I., and Sawada, Y. (2012). Dual inhibition of Src and GSK3 maintains mouse embryonic stem cells, whose differentiation is mechanically regulated by Src signaling. Stem Cells 30, 1394-1404.
Suchy, F. P., Nishimura, T., Wilkinson, A. C., Higuchi, M., Bhadury, J., and Nakauchi, H. (2020). Streamlined and quantitative detection of chimerism in mouse tissue using digital PCR. bioRxiv. doi.org/10.1101/2020.11.04. 368944.
Takebe, T., and Wells, J. M. (2019). Organoids by design. Science 364, 956-959.
Usui, J., Kobayashi, T., Yamaguchi, T., Knisely, A. S., Nishinakamura, R., and Nakauchi, H. (2012). Generation of kidney from pluripotent stem cells via blastocyst complementation. Am. J. Pathol. 180, 2417-2426.
Wada, J., Liu, Z. Z., Alvares, K., Kumar, A., Wallner, E., Makino, H., and Kanwar, Y. S. (1993). Cloning of cDNA for the alpha subunit of mouse insulin-like growth factor I receptor and the role of the receptor in metanephric development. Proc. Natl. Acad. Sci. USA 90, 10360-10364.
Wu, J., Platero-Luengo, A., Sakurai, M., Sugawara, A., Gil, M. A., Yamauchi, T., Suzuki, K., Bogliotti, Y. S., Cuello, C., Morales Valencia, M., et al. (2017). Interspecies chimerism with mammalian pluripotent stem cells. Cell 168, 473-486.e15.
Yamaguchi, T., Hamanaka, S., and Nakauchi, H. (2014). The generation and maintenance of rat induced pluripotent stem cells. In Stem Cells and Tissue Repair: Methods and Protocols, C. Kioussi, ed. (Springer), pp. 143-150.
Yamaguchi, T., Sato, H., Kato-Itoh, M., Goto, T., Hara, H., Sanbo, M., Mizuno, N., Kobayashi, T., Yanagida, A., Umino, A., et al. (2017). Interspecies organogenesis generates autologous functional islets. Nature 542, 191-196.
Yamaguchi, T., Sato, H., Kobayashi, T., Kato-Itoh, M., Goto, T., Hara, H., Mizuno, N., Yanagida, A., Umino, A., Hamanaka, S., et al. (2018). An interspecies barrier to tetraploid complementation and chimera formation. Sci. Rep. 8, 15289.
Yamanaka, S., Saito, Y., Fujimoto, T., Takamura, T., Tajiri, S., Matsumoto, K., and Yokoo, T. (2019). Kidney regeneration in later-stage mouse embryos via transplanted renal progenitor cells. J. Am. Soc. Nephrol. 30, 2293-2305.

### Experimental Model and Subject Details

### Animals

C57BL/6-Tg (UBC-GFP) 30Scha/J (RRID:IMSR_JAX:004353), C57BL/6J (RRID:IMSR_JAX:000664), and NSG (NOD.Cg-Prkdcscid ll2rgtm1Wjl/SzJ, RRID:IMSR_JAX:005557) mice were purchased from Jackson Laboratories (Bar Harbor, Me.: 004353, 000664, and 005557). For blastocyst injection experiments, three to seven-week-old CD1 female mice and 10-week-old male mice (RRI-D:IMSR_CRL:022) were purchased from Charles River Laboratory (Wilmington, Mass.). Littermates of the same sex were randomly assigned to experimental groups. All mice were housed in specific pathogen-free conditions with free access to food and water. All animal protocols were approved by the Administrative Panel on Laboratory Animal Care at Stanford University.

ESCs/iPSCs: Derivation and culture

Undifferentiated mouse ESCs (a2i/LIF2 line) were maintained in DMEM based-serum medium containing 1000 U/ml LIF (Peprotech, Cranbury, N.J.; 300-05), 1.5 μM Src kinase inhibitor CGP77675 (Sigma, St. Louis, Mo.; SML0314) and 3 μM GSK3 inhibitor CHIR99021 (Tocris, Barton Ln, Abingdon, United Kingdom; 4423) for a2i/LIF medium as described (Mori et al., 2019; Shimizu et al., 2012). Cells from the mESCs line (EB3DR line) were maintained as described (Yamaguchi et al., 2018). Undifferentiated rat iPSCs were maintained in N2B27 medium containing 1 μM MEK inhibitor PD0325901 (Tocris), 3 μM CHIR99021, and 1000 U/ml of rat LIF as described (Yamaguchi et al., 2014). Mouse ESCs (a2i/LIF2 line) were derived from C57BL/6-Tg (UBC-GFP) 30Scha/J mice. Their pluripotency was confirmed by chimera generation assay and by teratoma formation on injection into immunodeficient mice. The rat iPSCs ubiquitously express EGFP under the control of the ubiquitin-C promoter. All PSCs lines used in this study were male lines: We have not seen any sex difference in organ chimerism.

### Method Details

### Cas9 Ribonucleoprotein Electroporation

Cas9 ribonucleoproteins were introduced into zygotes of mice as described (Hashimoto et al., 2016; Mizuno et al., 2018). In brief, two-pronuclear zygotes were washed three times with Opti-MEM I medium (GIBCO, Waltham, Mass.). 20-30 zygotes were transferred into 5 μL Opti-MEM I medium containing 100 ng/μl Cas9 protein (IDT, Coralville, Iowa) and 100 ng/μl sgRNA (Synthego, Redwood City, Calif.) on LF501PT1-10 electrode (BEX, Tokyo, Japan). Electroporation was performed with Genome Editor (BEX) under the following conditions: 25 V, 3 ms ON, 97 ms OFF, Pd Alt 3 times. The sgRNA targets were: Igf1r sgRNA1, 5′-GAAAACTGCACGGTGATCGA-3′ (SEQ ID NO:1); sgRNA2, 5′-GGCCCCTGCCCCAAAGTCTG-3′ (SEQ ID NO:2).

### Flow Cytometry Analysis

Mouse peripheral blood cells were isolated from retroorbital sinus blood and stained with BV421-anti-CD45 antibody (Biolegend, San Diego, Calif.; 103134). Fetal blood cells obtained from the liver, aorta-gonad-mesonephros, and yolk sac were stained with BV421-anti-CD45 antibody. Donor chimerism was analyzed by detecting GFP-expressing cells. Analysis and sorting were performed by cytometry using FACS Aria II (BD, Franklin Lakes, N.J.).

Digital Droplet PCR (ddPCR) for Chimerism Analysis

Each organ or tissue (lung, liver, heart, brain, intestine, kidney, blood, gonad, thymus, and CT) was harvested from chimeric embryos at E10.5-12.5 and E18.5 and from adult chimeras after which DNA was extracted. Chimerism was determined by counting host and donor alleles with ddPCR as described below. Each reaction was prepared and analyzed with the QX200 ddPCR system (BioRad, Hercules, Calif.) in accordance with BioRad's standard recommendations for use with their ddPCR Supermix for Probes (No dUTP) unless otherwise stated. All reactions were 20 μl and contained up to 5 μl of extracted genomic DNA. BL6 (donor) versus CD1 (host) chimerism was analyzed using a digital PCR single-nucleotide discrimination assay (Hindson et al., 2011; Suchy et al., 2020). In brief, primers amplified a common region of the tyrosinase gene in CD1 and BL6 mouse (forward, mTyr-F/1, 1.8 uM AATAG-GACCTGCCAGTGCTC (SEQ ID NO:3); reverse, mTyr-R/1, 1.8 uM, TCAAGACTCGCTTCTCTGTACA (SEQ ID NO:4)), that differs by a single nucleotide between CD1 and BL6 mice. Two TaqMan probes with different fluorophores were used to detect either the CD1 mutant albino allele (mTyr-alb-P/1, 0.25 μM, fluorescein amidites (FAM)-cttaGagtttccgcagttgaaaccc (SEQ ID NO:5)-Black Hole Quencher [BHQ]) or the BL6 wild-type allele (mTyr-wt-P/1, 0.25 μM, hexachloro-fluorescein [HEX]-cttaCagtttccgcagttgaaaccc (SEQ ID NO:6)-BHQ) in a single reaction with the above primers. Fifty PCR cycles were run with 30 s melting at 94° C. and 1-minute (min) combined annealing/extension at 64° C. All reactions contained a total of 50-2000 copies/μl of the tyrosinase gene and at least 10,000 partitions.

For rat chimerism, primers and TaqMan probe were designed to detect a region of P53 that is specific to rat (forward, rP53-F/1, 0.9 μM, GGCAGGACAAAGAAGGTGGA (SEQ ID NO:7); reverse, rP53-R/1, 0.9 μM, GGGCAGTGCTATGGAAGGAG (SEQ ID NO:8); Probe, rP53-P/1, 0.25 μM, FAM-CGCCCTTCAGCTTCACCCCA (SEQ ID NO:9)-BHQ). Another set of primers and probe was designed to detect a genomic region identical in rat and mouse (forward, Zeb2-F/5, 0.9 μM, GGATGGGGAATGCAGCTCTT (SEQ ID NO:10); reverse, Zeb2-R/5.1, 0.9 uM, AGTGCGGCAGAATACAGCA (SEQ ID NO:11); Probe, 0.25 μM, Zeb2-P/5, HEX-TGATGGGTTGTGAAGGCAGCTGCACCT (SEQ ID NO:12)-BHQ). Both primer/probe sets were multiplexed in a single reaction. 50 PCR cycles were run with 30 s melting at 94° C. and 1-min combined annealing/extension at 60° C. The ratio of rP53:Zeb2 was used to determine percentages of chimerism. All reactions contained a total of 50-2000 copies/ul of Zeb2 and at least 10,000 partitions. Primers and probes were obtained from Sigma or IDT. Probes were labeled with either the FAM or HEX fluorophore at the 5′ end and with the BHQ quencher at the 3′ end.

### Embryo Culture and Manipulation

Wild-type mouse embryos were prepared according to published protocols (Brownstein, 2003; Mizuno et al., 2018). In brief, zygotes were obtained by oviduct perfusion from superovulated CD1 mice. Zygotes were cultured in KSOM-AA medium (CytoSpring, Mountain View, Calif.; K0101) for 1-4 hours and two-pronucleus zygotes were collected. Cas9 ribonucleoproteins were transfected by electroporation according to published protocols (Mizuno et al., 2018). After electroporation, zygotes were transferred to KSOM-AA medium and incubated for 3-5 days. For micromanipulation, ESCs or iPSCs were trypsinized and suspended in ESC or iPSC culture medium. A piezo-driven micromanipulator (Prime Tech, Tsuchiura, Japan) was used to drill the zona pellucida and trophectoderm under microscopy and 5-10 ESCs or iPSCs were introduced into blastocyst cavities near the inner cell mass. After blastocyst injection, embryos were cultured for 1-2 hours. Mouse blastocysts were then transferred into uteri of pseudopregnant recipient CD1 female mice (2.5 days post coitum). Table S3 shows results of the blastocyst injections.

### Genotyping

Host embryos were genotyped by PCR using crude lysate. Aliquots of lysate were incubated in 20 mM Tris-HCl (pH8.0, 100 mM NaCl, 5 mM EDTA, 0.1% SDS, 200 μg/mL proteinase K) at 60° C. for 5 minutes to 24 hours, followed by 98° C. proteinase K heat inactivation for 2 minutes. Genomic PCR was performed with SeqAmp DNA Polymerase (Takara Bio, Kusatsu, Japan) and these primers: mouse Igf1r sgRNA1 and 2, forward 5′-CAACCCTTTGTGACCTCGGA-3′ (SEQ ID NO:13), reverse 5′-AGAGGAAGAAAGCACGGAG-3′ (SEQ ID NO:14).

### Teratoma Formation

Approximately 1×10⁶ mESCs were injected subcutaneously into immunodeficient mice.

Four weeks later, the resultant tumor mass was excised. Hematoxylin and eosin-stained histologic sections were evaluated by light microscopy.

### Biochemical Assays in Serum

Serum levels of blood urea nitrogen, creatinine, and albumin were measured with routine techniques by Stanford Diagnostic Clinical Laboratory at Stanford University.

### Histological Analysis

Tissues were fixed with 4% paraformaldehyde and embedded in paraffin. Paraffin-embedded sections were deparaffinized with xylene and hydrated with graded ethanol. An autoclave was used for antigen retrieval. Sections were stained with hematoxylin and eosin for light microscopy. When immunostaining, each section was incubated with the primary antibody for 1-2 hours and with the secondary antibody for 1 hour, both at room temperature (detailed in Table S4). Following a wash step, sections were mounted with Fluoromount-G™, containing 4′,6-diamidino-2-phenylindole (DAPI; Invitrogen, Carlsbad, Calif.; 00-4959-52), and observed under fluorescence microscopy or confocal laser scanning microscopy. Diaminobenzidine development was performed with ImmPRESS Horse anti-Rabbit IgG PLUS Polymer Kit, peroxidase (Vector Laboratories, Burlingame, Calif.; MP-7801) according to the manufacturer's instructions.

### Quantification and Statistical Analysis

Upon log transformation, organ:blood ratios and % chimerism measurements achieved normal distribution, justifying the use of parametric statistics where indicated. Analysis of variance (ANOVA) was undertaken with CT:blood ratio as the dependent variable, and embryonic day (E10.5, E11.5, E12.5, and E18.5) and Igf1r genotype (WT and null) as two between-subjects factors. Analysis was performed with SPSS version 19 software. The few embryos with no chimerism (0%) or high blood chimerism (>40%) were excluded from analysis.

When n≥5, unpaired two tailed t tests were performed, as indicated in the figures, using Prism 7 software. When n<5, unpaired Mann-Whitney U non-parametric tests were performed, as indicated in the figures, using SPSS version 19 software. Chimerism and organ:blood ratios were log transformed if analyzed with a parametric test. Flow cytometry data was analyzed using FlowJo 10.6.2.

TABLE 1
Key Resources

| | | |
|---|---|---|
| REAGENT or RESOURCE | SOURCE | IDENTIFIER |

Antibodies

| | | |
|---|---|---|
| Rat anti-mouse CD45 antibody | Biolegend | Cat#103134; RRID:AB_2562559 |
| Chicken anti-GFP antibody | Abeam | Cat#ab13970; RRID:AB_300798 |
| Rabbit anti-rat AQP1 antibody | Millipore | Cat#AB2219; RRID:AB_1163380 |
| mouse anti-rabbit Na⁺/K⁺ ATPase | Millipore | Cat#05-369; RRID:AB_309699 |
| alpha-1 antibody | R&D | Cat#AF3159; RRID:AB_2155023 |
| Goat anti-mouse Nephrin antibody | | |
| Goat anti-mouse Podoplanin | R&D | Cat#AF3244; RRID:AB_2268062 |
| antibody | | |
| Rabbit anti-mouse N-Terminal Pro- | Seven Hills Bioreagents | Cat#WRAB-9337; |
| Surfactant Protein-C antibody | | RRID:AB_2335890 |
| Mouse anti-bovine Calbindin-D-28K | Sigma-Aldrich | Cat#C9848; RRID:AB_476894 |
| Antibody | Thermo Fisher Scientific | Cat#A-11035; RRID:AB_2534093 |
| Goat anti-rabbit IgG (H + L) Antibody | | |
| Donkey anti-goat IgG (H + L) | Thermo Fisher Scientific | Cat#A-11056; RRID:AB_142628 |
| Antibody | Thermo Fisher Scientific | Cat#A-21124; RRID:AB_2535766 |
| Goat anti-mouse lgG1 Antibody | | |
| Goat anti-chicken igY (H + L) | Thermo Fisher Scientific | Cat#A-11039; RRID:AB_2534096 |
| Antibody | | |

Experimental Models: Cell Lines

| | | |
|---|---|---|
| Mouse ESCs:EB3DR | Kobayashi et al., 2010 | N/A |
| Mouse ESCs:a2i/LIF2 | This paper | N/A |
| RatiPSCs:T1-3 | Yamaguchi et al., 2014 | N/A |

Experimental Models: Organisms/Strains

| | | |
|---|---|---|
| Mouse:C57bL/6J:C57BL/6- | The Jackson Laboratory | RRID:IMSR_JAX:004353 |
| Tg(UBC-GFP) 30Scha/J | | |
| Mouse: C57BL/6J | The Jackson Laboratory | RRID:IMSR_JAX:000664 |
| Mouse:NSG:NOD.Cg-Prkdc^{scid} | The Jackson Laboratory | RRID:IMSR_JAX:005557 |
| II2rg^{tm1Wj}I/SzJ | | |
| Mouse:Crl:CD1 (ICR) | Charles River Laboratories | RRID:IMSR_CRL:022 |

Oligonucleotides

| | | |
|---|---|---|
| Primers for ddPCR | This paper | N/A |
| Primers for genotyping | This paper | N/A |
| Probes for ddPCR | This paper | N/A |

Software and Algorithms

| | | |
|---|---|---|
| FlowJo | Tree Star flowjo.com | Version 10.6.2 |
| GraphPad Prism | GraphPad Software; | Version 8 |
| | graphpad.com/scientific- | |
| | software/prism | |
| SPSS software | ibm.com/support/pages/ibmspss- | version 19 |
| | statistics-19-documentation | |

TABLE 2
Organ chimerism of IGF1R chimera at E18.5
Chimerism (%)

| Neonate | Lung | Kidney | Blood | MEFs | Intestine |
|---|---|---|---|---|---|
| 1 | 19.5 | 47.0 | 8.7 | 25.3 | 21.7 |
| 2 | 45.6 | 54.4 | 8.8 | 30.1 | 21.5 |
| 3 | 27.7 | 35.8 | 3.5 | 17.1 | 4.9 |
| 4 | 38.3 | 40.3 | 15.1 | 29.8 | 25.8 |
| 5 | 72.1 | 77.7 | 25.5 | 61.9 | 56.4 |

### Example 2

### Deletion of Insulin Receptor Increases Donor Chimerism in Several Organs

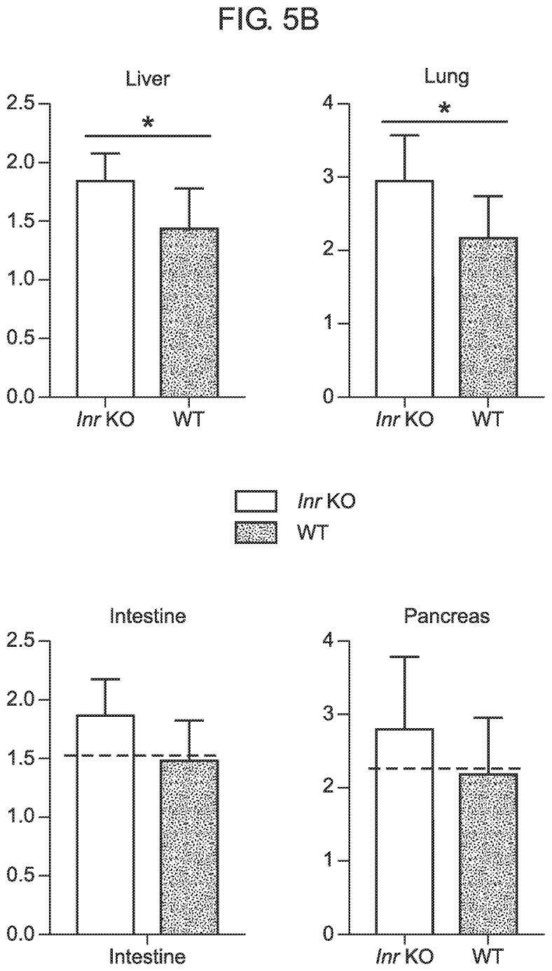

Insulin has a critical role in cell metabolism, and it has been known that insulin and Igf1 interact with each receptor to compensate or help the other function. In addition, insulin receptor (Inr) is highly homologous to the IGF1R, sharing 84% amino acid identity in the kinase domain and 100% conservation on the ATP binding pocket in human. Therefore, we investigated whether the deletion of Inr in a host embryo increases donor contribution to chimeric embryos. To generate chimeric embryos, mouse ESCs (mESCs) were injected into Inr null embryos and the donor chimerism was compared with the chimera generated by injecting mESCs into wild-type embryos. To validate donor chimerism increase, organ per blood chimerism ratio was used for the analysis. The deletion of Inr in host embryos slightly increased whole per blood chimerism ratio compared to that of wild-type chimeras (FIG. 5A). This result indicates that although Inr deletion increases donor chimerism, the extent of increase is not as large as Igf1r deletion. This is consistent with a previous report that Igf1r deletion decreases a neonatal body size to 45%, whereas Inr deletion induces just 10% of body size reduction. In addition, the organ per blood chimerism ratio was increased in liver, lung, intestine, and pancreas (FIG. 5B). Altogether, these results suggest that Inr deletion increases donor chimerism within rodent chimeric embryos as well as Igf1r deletion.

1. A method of creating a chimeric organ or tissue donor, the method comprising:
a) genetically modifying a non-human animal host embryo by deleting or inactivating a growth factor receptor gene; and
b) transplanting mammalian stem cells having a wild-type growth factor receptor gene into the non-human animal host embryo, wherein chimeric organs and tissue comprising mammalian cells are produced from the mammalian stem cells as the non-human animal host embryo grows.

2. The method of claim 1, wherein the growth factor receptor gene is an insulin-like growth factor 1 receptor (IGF1R) or an insulin receptor (INR) gene.

3. The method of claim 1, wherein the non-human animal is a vertebrate.

4. The method of claim 3, wherein the vertebrate is a mammal.

5. The method of claim 1, wherein the non-human host animal embryo is at the blastocyst stage or morula stage.

6. The method of claim 1, wherein the mammalian stem cells are embryonic stem cells, adult stem cells, or induced pluripotent stem cells.

7. The method of claim 1, wherein the mammalian stem cells are human stem cells.

8. The method of claim 1, wherein the mammalian stem cells are genetically modified to overexpress the growth factor receptor gene.

9. The method of claim 1, wherein said transplanting the mammalian stem cells is performed in utero to a conceptus or to the embryo in in vitro culture.

10. The method of claim 1, wherein said genetically modifying the non-human animal host embryo comprises using a clustered regularly interspaced short palindromic repeats (CRISPR) system, a transcription activator-like effector nuclease (TALEN), or a zinc-finger nuclease to delete or inactivate the growth factor receptor gene.

11. The method of claim 10, wherein the CRISPR system, TALEN, or zinc-finger nuclease is used to delete or introduce a frameshift mutation in at least one allele of the growth factor receptor gene.

12. The method of claim 11, wherein the CRISPR system, TALEN, or zinc-finger nuclease is used to delete or introduce a frameshift mutation in both alleles of the growth factor receptor gene.

13. The method of claim 10, wherein the CRISPR system targets an insulin-like growth factor 1 receptor (IGF1R) or insulin receptor (INR) gene or RNA transcript or makes epigenetic changes that reduce expression of the IGF1R or the INR gene.

14-15. (canceled)

16. A chimeric organ or tissue donor produced by the method of claim 1.

17. A method of transplanting an organ or tissue into a mammalian recipient subject, the method comprising transplanting a chimeric organ or tissue from the chimeric organ or tissue donor of claim 16 to the mammalian recipient subject.

18. The method of claim 17, wherein at least 90% of the cells in the chimeric organ or tissue are produced from the mammalian stem cells.

19. The method of claim 18, wherein the stem cells are human stem cells.

20. The method of claim 17, wherein the mammalian stem cells are adult stem cells from the mammalian recipient subject or induced pluripotent stem cells derived from cells from the mammalian recipient subject.

21-24. (canceled)

25. A non-human animal host embryo comprising:
a) a genetically modified genome comprising a knockout of an insulin-like growth factor 1 receptor (IGF1R) gene or an insulin receptor (INR) gene; and
b) transplanted mammalian stem cells having a wild-type growth factor receptor gene, wherein said non-human animal host embryo produces chimeric organs and tissue comprising mammalian cells from the mammalian stem cells during development.

26-29. (canceled)

30. The non-human animal host embryo of claim 25, wherein the mammalian stem cells are human stem cells.

31. The non-human animal host embryo of claim 25, wherein the mammalian stem cells are genetically modified to overexpress the IGF1R gene or the INR gene.

32-39. (canceled)


SEQUENCE LISTING (14 sequences)

SEQ ID NO: 1 - DNA, 20 bp, Artificial Sequence
  Igf1r sgRNA1
  source (1)..(20): sequence is synthesized
gaaaactgcacggtgatcga

SEQ ID NO: 2 - DNA, 20 bp, Artificial Sequence
  sgRNA2
  source (1)..(20): sequence is synthesized
ggcccctgccccaaagtctg

SEQ ID NO: 3 - DNA, 20 bp, Artificial Sequence
  mTyr-F/1 forward primer
  source (1)..(20): sequence is synthesized
aataggacctgccagtgctc

SEQ ID NO: 4 - DNA, 22 bp, Artificial Sequence
  mTyr-R/1 reverse primer
  source (1)..(22): sequence is synthesized
tcaagactcgcttctctgtaca

SEQ ID NO: 5 - DNA, 25 bp, Artificial Sequence
  CD1 mutant albino allele (mTyr-alb-P/1) probe
  source (1)..(25): sequence is synthesized
cttagagtttccgcagttgaaaccc

SEQ ID NO: 6 - DNA, 25 bp, Artificial Sequence
  BL6 wild-type allele (mTyr-wt-P/1) probe
  source (1)..(25): sequence is synthesized
cttacagtttccgcagttgaaaccc

SEQ ID NO: 7 - DNA, 20 bp, Artificial Sequence
  rP53-F/1 forward primer
  source (1)..(20): sequence is synthesized
ggcaggacaaagaaggtgga

SEQ ID NO: 8 - DNA, 20 bp, Artificial Sequence
  rP53-R/1 reverse primer
  source (1)..(20): sequence is synthesized
gggcagtgctatggaaggag

SEQ ID NO: 9 - DNA, 20 bp, Artificial Sequence
  rP53-P/1 probe
  source (1)..(20): sequence is synthesized
cgcccttcagcttcacccca

SEQ ID NO: 10 - DNA, 20 bp, Artificial Sequence
  Zeb2-F/5 forward primer
  source (1)..(20): sequence is synthesized
ggatggggaatgcagctctt

SEQ ID NO: 11 - DNA, 19 bp, Artificial Sequence
  Zeb2-R/5.1 reverse primer
  source (1)..(19): sequence is synthesized
agtgcggcagaatacagca

SEQ ID NO: 12 - DNA, 27 bp, Artificial Sequence
  Zeb2-P/5 probe
  source (1)..(27): sequence is synthesized
tgatgggttgtgaaggcagctgcacct

SEQ ID NO: 13 - DNA, 20 bp, Artificial Sequence
  mouse Igf1r forward primer
  source (1)..(20): sequence is synthesized
caaccctttgtgacctcgga

SEQ ID NO: 14 - DNA, 19 bp, Artificial Sequence
  mouse Igf1r reverse primer
  source (1)..(19): sequence is synthesized
agaggaagaaagcacggag